SEQUENCE LISTING (41 sequences)

SEQ ID NO: 1 - DNA, 1281 bp, Escherichia coli K12
atggctgatacaaaagcaaaactcaccctcaacggggatacagctgttgaactggatgtgctgaaaggcacgctgggtcaagatgttattgatatccgtactctcggttcaaaaggtgtgttcacctttgacccaggcttcacttcaaccgcatcctgcgaatctaaaattacttttattgatggtgatgaaggtattttgctgcaccgcggtttcccgatcgatcagctggcgaccgattctaactacctggaagtttgttacatcctgctgaatggtgaaaaaccgactcaggaacagtatgacgaatttaaaactacggtgacccgtcataccatgatccacgagcagattacccgtctgttccatgctttccgtcgcgactcgcatccaatggcagtcatgtgtggtattaccggcgcgctggcggcgttctatcacgactcgctggatgttaacaatcctcgtcaccgtgaaattgccgcgttccgcctgctgtcgaaaatgccgaccatggccgcgatgtgttacaagtattccattggtcagccatttgtttacccgcgcaacgatctctcctacgccggtaacttcctgaatatgatgttctccacgccgtgcgaaccgtatgaagttaatccgattctggaacgtgctatggaccgtattctgatcctgcacgctgaccatgaacagaacgcctctacctccaccgtgcgtaccgctggctcttcgggtgcgaacccgtttgcctgtatcgcagcaggtattgcttcactgtggggacctgcgcacggcggtgctaacgaagcggcgctgaaaatgctggaagaaatcagctccgttaaacacattccggaatttgttcgtcgtgcgaaagacaaaaatgattctttccgcctgatgggcttcggtcaccgcgtgtacaaaaattacgacccgcgcgccaccgtaatgcgtgaaacctgccatgaagtgctgaaagagctgggcacgaaggatgacctgctggaagtggctatggagctggaaaacatcgcgctgaacgacccgtactttatcgagaagaaactgtacccgaacgtcgatttctactctggtatcatcctgaaagcgatgggtattccgtcttccatgttcaccgtcattttcgcaatggcacgtaccgttggctggatcgcccactggagcgaaatgcacagtgacggtatgaagattgcccgtccgcgtcagctgtatacaggatatgaaaaacgcgactttaaaagcgatatcaagcgt

SEQ ID NO: 2 - DNA, 1281 bp, Artificial
  Corn-codon optimized sequence
atggccgacaccaaggccaagctgaccctgaacggcgacaccgccgtggagctggacgtgctgaagggcaccctgggccaggacgtgatcgacatcaggaccctgggcagcaagggcgtgttcaccttcgacccgggcttcaccagcaccgccagctgcgagagcaagatcaccttcatcgacggcgacgagggcatcctgctgcacaggggcttcccgatcgaccagctggccaccgacagcaactacctggaggtgtgctacatcctgctgaacggcgagaagccgacccaggagcagtacgacgagttcaagaccaccgtgaccaggcacaccatgatccacgagcagatcaccaggctgttccacgccttcaggagggacagccacccgatggccgtgatgtgcggcatcaccggcgccctggccgccttctaccacgacagcctggacgtgaacaacccgaggcacagggagatcgccgccttcaggctgctgagcaagatgccgactatggccgccatgtgctacaagtacagcatcggccagccgttcgtgtacccgaggaacgacctgagctacgccggcaacttcctgaacatgatgttcagcaccccgtgcgagccgtacgaggtgaacccgatcctggagagggcgatggacaggatcctgatcctgcacgccgaccacgagcagaacgccagcaccagcaccgtgaggaccgccggcagcagcggcgccaacccgttcgcctgcatcgccgccggcatcgccagcctgtggggcccggcccacggcggcgccaacgaggccgccctgaagatgctggaggagatcagcagcgtgaagcacatcccggagttcgtgaggagggccaaggacaagaacgacagcttcaggctgatgggcttcggccacagggtgtacaagaactacgacccgagggccaccgtgatgagggagacctgccacgaggtgctgaaggagctgggcaccaaggacgacctgctggaggtggctatggagctggagaacatcgccctgaacgacccgtacttcatcgagaagaagctgtacccgaacgtggacttctacagcggcatcatcctgaaggcgatgggcatcccgagcagcatgttcaccgtgatcttcgcgatggccaggaccgtgggctggatcgcccactggagcgagatgcacagcgacggcatgaagatcgccaggccgaggcagctgtacaccggctacgagaagagggacttcaagagcgacatcaagagg

SEQ ID NO: 3 - DNA, 1332 bp, Saccharomyces cerevisiae
atgagtagcgcctccgaacaaacgttgaaggagagatttgctgaaattatcccagcaaaggcacaagaaattaaaaaattcaagaaagaacacggtaaaaccgttattggtgaagttcttttggaggagcaagcttatggtggtatgagaggtattaaaggccttgtttgggaaggttccgtgttagaccccgaagaaggtattagatttaggggtcgtactattccagaaattcaaagggaactaccaaaggctgagggtagtacagaacctttgccagaagctttattttggttgcttttgactggtgaaatacctactgacgctcaagttaaagccctttctgctgatttagctgccagatcagaaattccagagcacgttatccaacttttagatagcctcccaaaagatctacatccaatggcgcaattttctattgccgtgactgctttagaaagcgagtctaagtttgccaaagcatatgctcaaggtgtatccaagaaagaatattggagctatacatttgaagattcgttagatctgctgggtaaattacctgttattgcttccaaaatttatcgtaatgtgttcaaggatggtaaaattacttcaaccgatcctaatgctgactatggtaaaaatttggcccaacttttgggctacgaaaacaaggattttattgacttaatgagactatatttaactattcattctgatcatgaaggtggtaacgtttctgcccatactacacatttagtgggttctgccttatcttcgccatacttatctttggccgctggtttgaatggtttagctggcccattacatggtcgtgccaatcaagaagttttagaatggctatttaaattgagagaagaagtgaaaggtgactattcaaaagaaacaattgaaaagtacttgtgggatactttgaacgcagggagagttgttcctggttatggccatgcggttttgagaaaaactgatcctcgttatacggctcaacgtgaattcgcattgaaacatttcccagattacgagttatttaagttggtctccaccatttatgaagttgccccaggggttttaactaagcatggtaaaactaagaacccatggccaaatgttgattcacattccggtgttttattgcaatactatggtctaactgaggcttcgttctacactgtattgtttggtgttgccagagctattggtgtgttaccccaattaatcatcgatagggctgttggtgctccaatcgaaaggccaaaatcattctccaccgaaaaatacaaggagttggtaaagaaaatcgaaagtaagaac

SEQ ID NO: 4 - DNA, 1383 bp, Artificial
  Corn-codon optimized sequence
atgagcgccatcctgagcaccaccagcaagagcttcctgagcaggggcagcaccaggcagtgccagaacatgcagaaggccctgttcgccctgctgaacgccaggcactacagcagcgccagcgagcagaccctgaaggagaggttcgccgagatcatcccggccaaggcccaggagatcaagaagttcaagaaggagcacggcaagaccgtgatcggcgaggtgctgctggaggagcaggcctacggcggcatgaggggcatcaagggcctggtgtgggagggcagcgtgctggacccggaggagggcatcaggttcaggggcaggaccatcccggagatccagagggagctgccgaaggccgagggcagcaccgagccgctgccggaggccctgttctggctgctgctgaccggcgagatcccgaccgacgcccaggtgaaggccctgagcgccgacctggccgccaggagcgagatcccggagcacgtgatccagctgctggacagcctgccgaaggacctgcacccgatggcccagttcagcatcgccgtgaccgccctggagagcgagagcaagttcgccaaggcctacgcccagggcgtgagcaagaaggagtactggagctacaccttcgaggacagcctggacctgctgggcaagctgccggtgatcgccagcaagatctacaggaacgtgttcaaggacggcaagatcaccagcaccgacccgaacgccgactacggcaagaacctggcccagctgctgggctacgagaacaaggacttcatcgacctgatgaggctgtacctgaccatccacagcgaccacgagggcggcaacgtgagcgcccacaccacccacctggtgggcagcgccctgagcagcccgtacctgagcctggccgccggcctgaacggcctggccggcccgctgcacggcagggccaaccaggaggtgctggagtggctgttcaagctgagggaggaggtgaagggcgactacagcaaggagaccatcgagaagtacctgtgggacaccctgaacgccggcagggtggtgccgggctacggccacgccgtgctgaggaagaccgacccgaggtacaccgcccagagggagttcgccctgaagcacttcccggactacgagctgttcaagctggtgagcaccatctacgaggtggccccgggcgtgctgaccaagcacggcaagaccaagaacccgtggccgaacgtggacagccacagcggcgtgctgctgcagtactacggcctgaccgaggccagcttctacaccgtgctgttcggcgtggccagggccatcggcgtgctgccgcagctgatcatcgacagggccgtgggcgccccgatcgagaggccg

SEQ ID NO: 5 - DNA, 1380 bp, Saccharomyces cerevisiae
atgacagttccttatctaaattcaaacagaaatgttgcatcatatttacaatcaaattcaagccaagaaaagactctaaaagagagatttagcgaaatctaccccatccatgctcaagatgtaaggcaattcgttaaagagcatggcaaaactaaaattagcgatgttctattagaacaggtatatggtggtatgagaggtattccagggagcgtatgggaaggttccgttttggacccagaagacggtattcgtttcagaggtcgtacgatcgccgacattcaaaaggacctgcccaaggcaaaaggaagctcacaaccactaccagaagctctcttttggttattgctaactggcgaggttccaactcaagcgcaagttgaaaacttatcagctgatctaatgtcaagatcggaactacctagtcatgtcgttcaacttttggataatttaccaaaggacttacacccaatggctcaattctctattgctgtaactgccttggaaagcgagtcaaagtttgctaaggcttatgctcaaggaatttccaagcaagattattggagttatacttttgaagattcactagacttgctgggtaaattgccagttattgcagctaaaatttatcgtaatgtattcaaagatggcaaaatgggtgaagtggacccaaatgccgattatgctaaaaatctggtcaacttgattggttctaaggatgaagatttcgtggacttgatgagactttatttaaccattcattcggatcacgaaggtggtaatgtatctgcacatacatcccatcttgtgggctcagcactatcatcaccttatctgtcccttgcatcaggtttgaacgggttggctggcccacttcatgggcgtgctaatcaagaagtactagaatggttatttgcacttaaagaagaggtaaatgatgactactctaaagatacgatcgaaaaatatttatgggatactctaaactcaggaagagtcattcccggttatggtcatgctgtgctaaggaaaactgatcctcgttatatggctcagcgtaagtttgccatggaccattttccagattatgaattattcaagttagtttcatcaatatacgaggtagcacctggcgtattgactgaacatggtaaaaccaaaaatccatggccaaatgtagatgctcactctggtgtcttattacaatattatggactaaaagaatcttctttctataccgttttatttggcgtttcaagggcatttggtattcttgctcaattgatcactgatagggccatcggtgcttccattgaaaggccaaagtcctattctactgagaaatacaaggaattggtcaaaaacattgaaagcaaacta

SEQ ID NO: 6 - DNA, 1380 bp, Artificial
  Corn-codon optimized sequence
atgaccgtgccgtacctgaacagcaacaggaacgtggccagctacctgcagagcaacagcagccaggagaagaccctgaaggagaggttcagcgagatctacccgatccacgcccaggacgtgaggcagttcgtgaaggagcacggcaagaccaagatcagcgacgtgctgctggagcaggtgtacggcggcatgaggggcatcccgggcagcgtgtgggagggcagcgtgctggacccggaggacggcatcaggttcaggggcaggaccatcgccgacatccagaaggacctgccgaaggccaagggcagcagccagccgctgccggaggccctgttctggctgctgctgaccggcgaggtgccgacccaggcccaggtggagaacctgagcgccgacctgatgagcaggagcgagctgccgagccacgtggtgcagctgctggacaacctgccgaaggacctgcacccgatggcccagttcagcatcgccgtgaccgccctggagagcgagagcaagttcgccaaggcctacgcccagggcatcagcaagcaggactactggagctacaccttcgaggacagcctggacctgctgggcaagctgccggtgatcgccgccaagatctacaggaacgtgttcaaggacggcaagatgggcgaggtggacccgaacgccgactacgccaagaacctggtgaacctgatcggcagcaaggacgaggacttcgtggacctgatgaggctgtacctgaccatccacagcgaccacgagggcggcaacgtgagcgcccacaccagccacctggtgggcagcgccctgagcagcccgtacctgagcctggccagcggcctgaacggcctggccggcccgctgcacggcagggccaaccaggaggtgctggagtggctgttcgccctgaaggaggaggtgaacgacgactacagcaaggacaccatcgagaagtacctgtgggacaccctgaacagcggcagggtgatcccgggctacggccacgccgtgctgaggaagaccgacccgaggtacatggcccagaggaagttcgcgatggaccacttcccggactacgagctgttcaagctggtgagcagcatctacgaggtggccccgggcgtgctgaccgagcacggcaagaccaagaacccgtggccgaacgtggacgcccacagcggcgtgctgctgcagtactacggcctgaaggagagcagcttctacaccgtgctgttcggcgtgagcagggccttcggcatcctggcccagctgatcaccgacagggccatcggcgccagcatcgagaggccgaagagctacagcaccgagaagtacaaggagctggtgaagaacatcgagagcaagctg

SEQ ID NO: 7 - DNA, 1134 bp, Anabaena sp. PCC 7120
atgatggtgtgcgaatacaagcctggtttagaaggcattcccgccgcccaatcgagtatcagttatgtagatgggcaaaagggaatactagaatatcgtggcatccggattgaggatttagcccagcaaagtacttttctggagactgcttatcttttaatctggggtgagttgccaacaaaagaagaattgcaagtatttgaggaggaagtccgtcttcatcggcggattaaataccggattcgggatatgatgaagtgctttcccgaatctggtcatccaatggatgcactccaagcctctgcggcggctttaggcttgttttactcccgtcgagatttgcacaatcctgcctatattcgggatgctgtagtgcggctaatagctactattccgacgatggtagctgcattccagttgatgcggaaaggtaatgaccccgttaagccccgtgatgatttagattattccgccaattttctctacatgctcaacgagaaagaaccggatgctttggcggcaaaaatctttgatatctgcttgattctccatgtcgagcatacgatgaatgcttccacctttagtgctagggtaacagcttccaccttgactgacccgtatgcggtggttgctagcgctgtggggactttaggagggcctttacacggtggagccaatgaagaagtaatccagatgttggaagagattggttccgtggagaatgtgcgttcttatgtcgaggagaggttgcaacgtaaagacaagctcatgggctttggacatcgtgtctacaaagttaaagacccacgggcgacaattttgcaaggcctcgcagaacagttgtttgccaagttcggcgcagataagtattacgacatcgcccaagaaatggaacgggtagtcgaagagaaacttggtcataaagggatttatcccaatgttgacttctactctggtttagtgtatcggaagatgggtattcctacagacttgtttacaccaatctttgcgatcgctcgtgttgctggttggttagcccactggaaagaacaactcgaagagaaccgcattttccgtcctacccaggtttacaacggcaaacacagtgttacctacacccccattgaccaacgt

SEQ ID NO: 8 - DNA, 1134 bp, Artificial
  Corn-codon optimized sequence
atgatggtctgtgagtacaaacctggacttgaagggattcctgccgctcagtcgtctataagctacgttgacggtcaaaaaggcatacttgaataccgtggtatcagaattgaagaccttgcacaacaatcaactttcctcgaaactgcctacctcctcatctggggtgaactgccaaccaaggaagaattgcaagtttttgaagaagaagttcgcctccacagaagaattaagtaccgtataagagatatgatgaaatgcttccccgaatcaggccatcctatggatgctctccaagcctccgctgccgcccttggactcttctattcacgtcgcgacttgcataatccggcttacataagagatgcagttgtccgcctcatcgccacgattcctactatggttgctgccttccaactgatgagaaaagggaatgatcctgtgaagccccgtgatgatcttgactactccgcaaacttcttgtatatgcttaatgaaaaggaaccagacgctctcgctgctaaaatatttgatatttgtcttatcctccacgttgaacacaccatgaatgcatctacgttctccgctagagttactgccagcactcttaccgatccatacgccgttgttgcatccgctgtcggcactcttggtggcccactgcacggaggagcaaatgaagaagtcatccaaatgctcgaggagatcggctccgtcgaaaatgtacgtagttatgtcgaagaacgcctgcaaagaaaagacaagttgatgggattcggacatcgtgtatataaggtgaaagacccgcgtgcgactatcctgcagggcctggccgaacaactcttcgcaaaatttggagctgataaatactatgacatcgcacaggagatggaaagagtcgttgaggaaaaacttggtcataaaggtatctatccgaacgttgatttttactctggcctcgtttaccggaaaatgggcattcctactgacctgttcaccccgattttcgctatagctcgtgtcgctggctggctcgcccactggaaggaacaacttgaagaaaatcgcatttttagaccgacccaagtatacaatggaaagcactctgtaacttacacacccatagatcaacgg

SEQ ID NO: 9 - DNA, 1422 bp, Cucurbita cv. Kurokawa Amakuri
atgtcagctcagaccatggttgcgccgcctgaattggtgaagggtacgttgacgattgtagatgagagaactggaaagaggtaccaggtccaggtatctgaagaaggcacgatcaaggccaccgatttgaagaagataactacaggaccaaatgacaaggggcttaagctgtatgatccaggctatctcaacactgctccagttcggtcgtcgatcagttatattgatggtgacttgggaattcttaggtacagaggctacccgattgaggaattggctgagagtagtacctatgtggaagttgcatacctcttgatgtatgggaatttgccttctcagagtcaattggcagactgggaatttgctatttctcagcattcggctgtaccgcagggacttgtggatattattcaagcaatgcctcatgatgcacatccaatgggtgtgcttgttagtgcaatgagtgctctatctgtctttcatccagatgccaatcctgcccttagaggacaagatctttacaagtctaagcaagtgagagacaaacaaatagctcgtattatagggaaggctcccaccattgcagcagcagcttatcttagacttgctggaagacctccagttctcccttccagcaatctttcttattcggagaatttcctgtacatgcttgattctttgggtaataggtcttacaaacccaatcctcggcttgctagagtcctcgacattctattcatccttcatgcagaacatgaaatgaactgctcaacatctgctgctcgccatctggcttcaagtggtgttgatgtgttcactgctctttctggagctgtcggagcactgtatggccctcttcatggtggggccaatgaggctgtgcttaaaatgctaagtgagattggaactgttaataatattccagaattcatcgagggtgttaaaaacaggaaaaggaagatgtcaggttttggccatagggtttacaagaactacgatccaagagctaaggttataagaaaacttgccgaagaagtgttttccattgttggtcgggatcctctcattgaggtggctgttgctctggagaaggctgctctttcagatgagtattttgtcaagaggaaattatacccaaacgttgacttttactccggattaatatatagggctatgggatttccacctgaatttttcactgtgctgtttgcaatccctcgaatggctggatacttggcacattggcgagaatcgctggatgatcccgacactaagataattcgacctcaacaggtctacactggggaatggctgcgacattatataccacccaacgaacgacttgtaccggccaaggcagacaggcttggtcaggtttccgtttccaacgcctccaaacgccgattgtctggatcggggatc

SEQ ID NO: 10 - DNA, 1422 bp, Artificial
  Corn-codon optimized sequence
atgtctgctcagacaatggtcgccccccccgaactcgtcaaaggtacccttacaatcgtagacgaacgcacaggaaaaagataccaggtacaagtctcagaagaaggtactatcaaggccactgatcttaaaaaaattactactggtccaaatgacaaaggcttgaaactctatgatcccggttacttgaacaccgcaccagtccgctcttccatttcgtatattgatggtgatctcggcattcttagataccgcggatatcctattgaggagcttgcagaatcgtctacctacgtcgaagtagcatacttgctcatgtacggaaacctcccttcacagtcacaactggcagattgggaatttgctatatcacagcacagtgcagttccacaaggtttggtcgacataatccaggctatgccccatgacgcccaccctatgggagtcctcgtctctgctatgtctgccctttctgtattccaccctgatgctaacccagccttgcgtggccaagacctctacaaatccaaacaagttcgcgacaaacaaatagcacgcattattggcaaagcaccaacaattgcagccgccgcgtatcttaggctcgctggaaggcctcccgtgcttccgtcctctaacctgtcatattctgaaaacttcctgtacatgctcgactcactcggcaatagatcatacaagccgaatccacgcttggcaagggtcctcgacattctgttcattctccacgcagaacatgagatgaattgctcgacaagcgcagctagacatttggcatcatccggagtggatgtttttacagcattgtcaggcgccgtcggagccctttatggcccgctgcatggcggtgccaacgaagcggtcctcaaaatgctctcagagattggaacagtcaataatatacccgagttcattgaaggtgtaaagaacaggaagcgtaaaatgtcgggctttggccatagagtgtataaaaactatgacccgagagcaaaggtgattagaaagctcgccgaagaggttttctcaattgtaggacgcgatccccttattgaagttgctgttgcccttgaaaaggctgccctctcggacgaatatttcgtgaagcgcaaactctaccctaatgtcgatttttactcgggactcatttatcgcgcaatgggcttccctcctgaatttttcacagtactcttcgctatccctagaatggctggctacctcgcacattggagagaatctttggacgaccctgacactaagatcatcagaccacaacaagtatatactggggaatggcttagacactatatacctcctaacgaaaggctcgtgccggcaaaagctgacaggctcggtcaggtatccgttagcaatgcatccaaaagaagactctccgggtccggtatt

SEQ ID NO: 11 - DNA, 1548 bp, Artificial
  Corn-codon optimized sequence
atgccaaccgatatggaactttctccctcaaatgttgcaagacacagactcgcagtacttgccgcccatctcagcgctgcatcccttgaacctccagtcatggcctcatcccttgaagcccactgtgtttctgctcagacaatggtcgccccccccgaactcgtcaaaggtacccttacaatcgtagacgaacgcacaggaaaaagataccaggtacaagtctcagaagaaggtactatcaaggccactgatcttaaaaaaattactactggtccaaatgacaaaggcttgaaactctatgatcccggttacttgaacaccgcaccagtccgctcttccatttcgtatattgatggtgatctcggcattcttagataccgcggatatcctattgaggagcttgcagaatcgtctacctacgtcgaagtagcatacttgctcatgtacggaaacctcccttcacagtcacaactggcagattgggaatttgctatatcacagcacagtgcagttccacaaggtttggtcgacataatccaggctatgccccatgacgcccaccctatgggagtcctcgtctctgctatgtctgccctttctgtattccaccctgatgctaacccagccttgcgtggccaagacctctacaaatccaaacaagttcgcgacaaacaaatagcacgcattattggcaaagcaccaacaattgcagccgccgcgtatcttaggctcgctggaaggcctcccgtgcttccgtcctctaacctgtcatattctgaaaacttcctgtacatgctcgactcactcggcaatagatcatacaagccgaatccacgcttggcaagggtcctcgacattctgttcattctccacgcagaacatgagatgaattgctcgacaagcgcagctagacatttggcatcatccggagtggatgtttttacagcattgtcaggcgccgtcggagccctttatggcccgctgcatggcggtgccaacgaagcggtcctcaaaatgctctcagagattggaacagtcaataatatacccgagttcattgaaggtgtaaagaacaggaagcgtaaaatgtcgggctttggccatagagtgtataaaaactatgacccgagagcaaaggtgattagaaagctcgccgaagaggttttctcaattgtaggacgcgatccccttattgaagttgctgttgcccttgaaaaggctgccctctcggacgaatatttcgtgaagcgcaaactctaccctaatgtcgatttttactcgggactcatttatcgcgcaatgggcttccctcctgaatttttcacagtactcttcgctatccctagaatggctggctacctcgcacattggagagaatctttggacgaccctgacactaagatcatcagaccacaacaagtatatactggggaatggcttagacactatatacctcctaacgaaaggctcgtgccggcaaaagctgacaggctcggtcaggtatccgttagcaatgcatccaaaagaagactctccgggtccggtatt

SEQ ID NO: 12 - DNA, 1416 bp, Oryza sativa
atggcgttcttcaggggcctgaccgcggtgtcgaggcttcgatcccgcgtggcacaggaggccaccacgcttggtggtgtgcgatggctgcagatgcagagcgcatctgatcttgatctcaagtcccagctgcaggaattgattcctgaacaacaggaccgcttaaagaaacttaaatcggagcatggaaaggtccaacttggaaatataacagtcgatatggtccttggtgggatgagagggatgactggaatgctttgggaaacatcattgcttgacccggatgagggtattcgttttaggggtctctcgattccagagtgccagaaagtgctgccgacagcagttaaagatggggagcctttgcctgagggtctactttggcttcttttgaccggaaaggtgccaaccaaagagcaagttgatgctctatcaaaggaattggctagtcgttcgagtgttccaggtcatgtgtataaggcaatcgatgctctccctgtaactgctcatccgatgacccagtttaccacaggagtgatggcacttcaggtggagagtgagtttcaaaaagcctatgacaaaggaatgtcaaaatcaaagttctgggagcctacctatgaagattgcttaaatttgatagctcgccttccagcagtggcttcatatgtttaccggaggatattcaagggagggaaaactatagcagctgataatgcactggattatgcagcaaatttttcacacatgcttgggtttgatgatcccaaaatgcttgagttgatgcgactatatataacaatccacactgatcatgaaggtggaaacgtcagtgctcatactggacatctggttggaagtgctctgtcagacccttatctttcttttgcagctgcactgaatggtttagctggaccgttgcacggcctggctaatcaggaagtgttgttgtggatcaaatctgtaataggtgagactggtagtgacgttacaactgatcaactcaaagagtatgtgtggaagacactaaaaagtggaaaggttgttcctggctttggtcatggagttctacgtaagaccgatccacggtatacatgtcagagggagtttgctttgaagtacttgcctgaggatccacttttccaactggtctccaagttgtatgaagttgtgcctcctatcctcactgagcttggcaaggtcaaaaacccatggcctaatgttgatgctcacagcggagttctactgaaccactttggattatctgaagctcggtattacactgttcttttcggagtttcaaggagcattggaataggatctcagctcatttgggaccgtgctcttggcctgccgctcgaaagaccgaagagtgtcaccatggagtggctggagaaccactgcaagaaggttgctgct

SEQ ID NO: 13 - DNA, 1500 bp, Oryza sativa
atggatcgcgcccgcctcgccgtgctctccgcccacctcgcctcccccgccgccgcctgcggggaggcggacgcggcggggccgctggagaggtcggcggcgtctgcgggggcgcgaggcggcgcgctggcggtggtggatgggaggacggggaagaagtacgaggtcaaggtgtcggacgaggggaccgtgcacgccaccgacttcaagaagattaccactggaaaggacgacaagggtcttaagatctatgatcctggttatcccaacacagccccagttcgctcatccatctgctacattgatggggatgagggaattcttcgttacaggggatacccaattgaagagttggctgaaagcagctcatttgttgaggtggcctacctcctgatgtacggaagtttgcctacccagagccaattggctggatgggaatttgcgatttctcagcactctgctgttccccagggactcttggatatcatacaagcaatgcctcatgacgctcatcccatgggtgcccttgccagtgcaatgagcacgctttctgtcttccatccggatgcaaaccctgctcttagaggtcaagatctttacaagtcgaagcaggttagggataagcagattgtgcgagtacttgggaaggcaccaacaattgcagctgcagcgtacttgagattagctggaagacctcctatccttcctacaaatagtctctcttattcagagaacttcttgtatatgctagactctttgggtgacaaagaatacaagccaaatcttagacttgctagggttctagatatcctttttattctccatgctgaacatgaaatgaactgctctacagccgctgctaggcaccttgcttcaagtggtgttgatgtcttcactgctctttctggtgctggtggagctctatatggtccactgcatggtggtgcaaatgaggcggtacttaaaatgttaaatgaaattggaagtgtggagaatattccagatttcatcgagggagtgaaaaacaggaagagaaagatgtcaggttttgggcaccgtgtttacaagaattatgatccccgtgctaaagtcatccgaaagctagcagaggaggtcttctctattgtcggacgggatcctcttatcgaggttgctgttgcgttggagaaggcagcattgtcagatgattattttgtcaagaggaagctgtatccaaatgtggatttttactctggcttaatatatagggcaatgggattccctacagagttcttccctgttctgtttgcaattcctcgcatggctggttggttagcacattggaaagagtcacttgatgatccagacactaagattatgaggcctcagcaggtatacactggtgtttggctgaggcattacacacctgtcagagaacgagtcccagcaagccagggcgaacagcttggtcagattgctacctctaacgcaacaaggcgtcggcgtgcaggttctgccctg

SEQ ID NO: 14 - DNA, 1416 bp, Zea mays
atggcgttctatcggggcctcaccgcagtctcgagactgcgatcacgcatggcgcaggaggccaccacgctggggggtgtgaggtggctgcagatgcagagcgcgtccgatctcgatcttaagtcccagttgcaggaattgattccggaacaacaggatcgcttaaagaagctcaagtcagagcatggaaagacccagcttggaaacataactgtggatatggtccttggtggaatgagagggatgactggaatgctttgggaaacatccctacttgatccagaggagggtattcgttttaggggcctctcaattccagaatgccaaaaagtgctgccaacagcagttaagggcggtgaacctttgcctgagggtctcctttggcttcttttgacggggaaggtcccaaccaaagagcaagttgatgctctatcaaaggaattgcttgcgcgctcaactgtcccagctcatgtctataaggcaatagatgctctcccagtaactgcacatcctatgacacagtttaccacgggagtaatggctcttcaggttgagagcgaatttcaaaaagcttatgacaatggattgccaaaatcaaagttttgggagcctacttatgaagactgcttaaacttgattgctcggcttccaccagtggcttcttatgtttaccggagaattttcaagggtgggaaatcaatagaagccgataattctttggactatgcggcaaatttctcacacatgcttggttttgacgacccaaaaatgctggagctgatgcggctctatgtaacaattcacactgatcatgaaggcgggaatgtcagtgctcatactggtcatctggttggaagtgctctgtcagatccttatctttctttcgcagcggctctaaatgggttagctgggccactacatggccttgcaaatcaggaagtgcttttatggatcaaatctgtaattcaggaaactgggagtgatgttacaacggatcaactcaaagactatgtctggaagacactaaagagtggaaaggttgttcctgggtttggtcatggagttctgcgtaagaccgacccacggtattcatgtcaaagggagtttgccctgaagcatttgcccgaggatccacttttccaattggtgtccaagttgtatgaagttgtacctcctatcctcactgagctgggcaaggtcaagaacccatggccaaatgttgatgctcacagtggagttttgctgaaccactttggactatctgaagcacggtattacactgtcttgttcggtgtttcaagaagcatggggataggatctcagctcatctgggaccgtgcccttggcctgccacttgagaggccgaagagtgttaccatggagtggctggagaactactgcaagaacaaggctgct

SEQ ID NO: 15 - DNA, 1509 bp, Zea mays
atggatcgcgccgaccccgcgcggggccgccttgccgtgctctcctcccacctccgtggtgcaggggccgaggaggcggcggggctggagaggtcgccggtatccgcgccggcgcccgggccccgcgccggcgcgcttgccgtggtggacgggaggaccgggaagcggcacgaggtcaaggtctccgaagacggcaccgtgcgcgccaccgacttcaagaagattaccactggaaaggacgacaagggtcttaagatttatgatcctggttaccttaacactgcccctgttcgctcgtccatctgctacatcgatggagatgagggaatccttcgctataggggttatccaatcgaagaattggctgaaagcagctcgtttgttgaggtggcctaccttttaatgtatgggaacttgcctactcagagtcaattggcaggctgggaatttgctatttctcagcattctgctgttccccaaggactgttggatatcatacaatcaatgccccatgatgctcaccccatgggtgttcttgccagtgctatgagcaccctttctgtctttcatccagatgcaaaccctgctctacaaggtcaagatctttataaatcgaagcaggtgagggataaacaaattgtgcgagtacttgggaaggcaccaacaatagcagctgctgcctacttgagattagcaggaagacctcccgtccttcctttaaatactctatcttattcagagaacttcttgtacatgctggactctttgggtgacagaacatataaaccaaatcctcgacttgctcgagctctagatattctttttattctgcatgctgaacatgaaatgaactgctccactgctgctgttaggcaccttgcttcaagtggtgtggatgtatttactgctctttctggtggtgttggagctctatatggtcctctgcatggcggcgcaaacgaggcagtacttaaaatgttaaatgagattggaagcatggaaaatattccagatttcattgtaggagtgaagaacaggaagaggaagatgtccggttttgggcaccgtgtgtataaaaactatgaccctcgtgctaaagtcataaggaaattggcagatgaggtgttctcaattgttggacgggatccacttattgaggtggccattgccctagaaaaggcagcgctgtcagacgaatattttatcaagaggaagctgtatccaaatgtggatttctactctgggctaatttatagggcaatgggattccctacagaattttttcctgtgctgtttgctattcctcgcatgggtggctggctagcgcattggaaagagtcactcgatgatcctgacactaagattataaggccccaacaggtatacaccggcttctggcttaggcactatacccccgtcagagaacgagtgctatcaagccagagtgaggaacttggtcaggttgccacctcaaacgcaactaggcgccgccgtgctggttctgccctg

SEQ ID NO: 16 - protein, 427 aa, Escherichia coli K12
MADTKAKLTLNGDTAVELDVLKGTLGQDVIDIRTLGSKGVFTFDPGFTSTASCESKITFIDGDEGILLHRGFPIDQLATDSNYLEVCYILLNGEKPTQEQYDEFKTTVTRHTMIHEQITRLFHAFRRDSHPMAVMCGITGALAAFYHDSLDVNNPRHREIAAFRLLSKMPTMAAMCYKYSIGQPFVYPRNDLSYAGNFLNMMFSTPCEPYEVNPILERAMDRILILHADHEQNASTSTVRTAGSSGANPFACIAAGIASLWGPAHGGANEAALKMLEEISSVKHIPEFVRRAKDKNDSFRLMGFGHRVYKNYDPRATVMRETCHEVLKELGTKDDLLEVAMELENIALNDPYFIEKKLYPNVDFYSGIILKAMGIPSSMFTVIFAMARTVGWIAHWSEMHSDGMKIARPRQLYTGYEKRDFKSDIKR

SEQ ID NO: 17 - protein, 444 aa, Saccharomyces cerevisiae
MSSASEQTLKERFAEIIPAKAQEIKKFKKEHGKTVIGEVLLEEQAYGGMRGIKGLVWEGSVLDPEEGIRFRGRTIPEIQRELPKAEGSTEPLPEALFWLLLTGEIPTDAQVKALSADLAARSEIPEHVIQLLDSLPKDLHPMAQFSIAVTALESESKFAKAYAQGVSKKEYWSYTFEDSLDLLGKLPVIASKIYRNVFKDGKITSTDPNADYGKNLAQLLGYENKDFIDLMRLYLTIHSDHEGGNVSAHTTHLVGSALSSPYLSLAAGLNGLAGPLHGRANQEVLEWLFKLREEVKGDYSKETIEKYLWDTLNAGRVVPGYGHAVLRKTDPRYTAQREFALKHFPDYELFKLVSTIYEVAPGVLTKHGKTKNPWPNVDSHSGVLLQYYGLTEASFYTVLFGVARAIGVLPQLIIDRAVGAPIERPKSFSTEKYKELVKKIESKN

SEQ ID NO: 18 - protein, 460 aa, Saccharomyces cerevisiae
MTVPYLNSNRNVASYLQSNSSQEKTLKERFSEIYPIHAQDVRQFVKEHGKTKISDVLLEQVYGGMRGIPGSVWEGSVLDPEDGIRFRGRTIADIQKDLPKAKGSSQPLPEALFWLLLTGEVPTQAQVENLSADLMSRSELPSHVVQLLDNLPKDLHPMAQFSIAVTALESESKFAKAYAQGISKQDYWSYTFEDSLDLLGKLPVIAAKIYRNVFKDGKMGEVDPNADYAKNLVNLIGSKDEDFVDLMRLYLTIHSDHEGGNVSAHTSHLVGSALSSPYLSLASGLNGLAGPLHGRANQEVLEWLFALKEEVNDDYSKDTIEKYLWDTLNSGRVIPGYGHAVLRKTDPRYMAQRKFAMDHFPDYELFKLVSSIYEVAPGVLTEHGKTKNPWPNVDAHSGVLLQYYGLKESSFYTVLFGVSRAFGILAQLITDRAIGASIERPKSYSTEKYKELVKNIESKL

SEQ ID NO: 19 - protein, 378 aa, Anabaena sp. PCC 7120
MMVCEYKPGLEGIPAAQSSISYVDGQKGILEYRGIRIEDLAQQSTFLETAYLLIWGELPTKEELQVFEEEVRLHRRIKYRIRDMMKCFPESGHPMDALQASAAALGLFYSRRDLHNPAYIRDAVVRLIATIPTMVAAFQLMRKGNDPVKPRDDLDYSANFLYMLNEKEPDALAAKIFDICLILHVEHTMNASTFSARVTASTLTDPYAVVASAVGTLGGPLHGGANEEVIQMLEEIGSVENVRSYVEERLQRKDKLMGFGHRVYKVKDPRATILQGLAEQLFAKFGADKYYDIAQEMERVVEEKLGHKGIYPNVDFYSGLVYRKMGIPTDLFTPIFAIARVAGWLAHWKEQLEENRIFRPTQVYNGKHSVTYTPIDQR

SEQ ID NO: 20 - protein, 474 aa, Cucurbita cv. Kurokawa Amakuri
MSAQTMVAPPELVKGTLTIVDERTGKRYQVQVSEEGTIKATDLKKITTGPNDKGLKLYDPGYLNTAPVRSSISYIDGDLGILRYRGYPIEELAESSTYVEVAYLLMYGNLPSQSQLADWEFAISQHSAVPQGLVDIIQAMPHDAHPMGVLVSAMSALSVFHPDANPALRGQDLYKSKQVRDKQIARIIGKAPTIAAAAYLRLAGRPPVLPSSNLSYSENFLYMLDSLGNRSYKPNPRLARVLDILFILHAEHEMNCSTSAARHLASSGVDVFTALSGAVGALYGPLHGGANEAVLKMLSEIGTVNNIPEFIEGVKNRKRKMSGFGHRVYKNYDPRAKVIRKLAEEVFSIVGRDPLIEVAVALEKAALSDEYFVKRKLYPNVDFYSGLIYRAMGFPPEFFTVLFAIPRMAGYLAHWRESLDDPDTKIIRPQQVYTGEWLRHYIPPNERLVPAKADRLGQVSVSNASKRRLSGSGI

SEQ ID NO: 21 - protein, 516 aa, Cucurbita cv. Kurokawa Amakuri
MPTDMELSPSNVARHRLAVLAAHLSAASLEPPVMASSLEAHCVSAQTMVAPPELVKGTLTIVDERTGKRYQVQVSEEGTIKATDLKKITTGPNDKGLKLYDPGYLNTAPVRSSISYIDGDLGILRYRGYPIEELAESSTYVEVAYLLMYGNLPSQSQLADWEFAISQHSAVPQGLVDIIQAMPHDAHPMGVLVSAMSALSVFHPDANPALRGQDLYKSKQVRDKQIARIIGKAPTIAAAAYLRLAGRPPVLPSSNLSYSENFLYMLDSLGNRSYKPNPRLARVLDILFILHAEHEMNCSTSAARHLASSGVDVFTALSGAVGALYGPLHGGANEAVLKMLSEIGTVNNIPEFIEGVKNRKRKMSGFGHRVYKNYDPRAKVIRKLAEEVFSIVGRDPLIEVAVALEKAALSDEYFVKRKLYPNVDFYSGLIYRAMGFPPEFFTVLFAIPRMAGYLAHWRESLDDPDTKIIRPQQVYTGEWLRHYIPPNERLVPAKADRLGQVSVSNASKRRLSGSGI

SEQ ID NO: 22 - protein, 472 aa, Oryza sativa
MAFFRGLTAVSRLRSRVAQEATTLGGVRWLQMQSASDLDLKSQLQELIPEQQDRLKKLKSEHGKVQLGNITVDMVLGGMRGMTGMLWETSLLDPDEGIRFRGLSIPECQKVLPTAVKDGEPLPEGLLWLLLTGKVPTKEQVDALSKELASRSSVPGHVYKAIDALPVTAHPMTQFTTGVMALQVESEFQKAYDKGMSKSKFWEPTYEDCLNLIARLPAVASYVYRRIFKGGKTIAADNALDYAANFSHMLGFDDPKMLELMRLYITIHTDHEGGNVSAHTGHLVGSALSDPYLSFAAALNGLAGPLHGLANQEVLLWIKSVIGETGSDVTTDQLKEYVWKTLKSGKVVPGFGHGVLRKTDPRYTCQREFALKYLPEDPLFQLVSKLYEVVPPILTELGKVKNPWPNVDAHSGVLLNHFGLSEARYYTVLFGVSRSIGIGSQLIWDRALGLPLERPKSVTMEWLENHCKKVAA

SEQ ID NO: 23 - protein, 500 aa, Oryza sativa
MDRARLAVLSAHLASPAAACGEADAAGPLERSAASAGARGGALAVVDGRTGKKYEVKVSDEGTVHATDFKKITTGKDDKGLKIYDPGYPNTAPVRSSICYIDGDEGILRYRGYPIEELAESSSFVEVAYLLMYGSLPTQSQLAGWEFAISQHSAVPQGLLDIIQAMPHDAHPMGALASAMSTLSVFHPDANPALRGQDLYKSKQVRDKQIVRVLGKAPTIAAAAYLRLAGRPPILPTNSLSYSENFLYMLDSLGDKEYKPNLRLARVLDILFILHAEHEMNCSTAAARHLASSGVDVFTALSGAGGALYGPLHGGANEAVLKMLNEIGSVENIPDFIEGVKNRKRKMSGFGHRVYKNYDPRAKVIRKLAEEVFSIVGRDPLIEVAVALEKAALSDDYFVKRKLYPNVDFYSGLIYRAMGFPTEFFPVLFAIPRMAGWLAHWKESLDDPDTKIMRPQQVYTGVWLRHYTPVRERVPASQGEQLGQIATSNATRRRRAGSAL

SEQ ID NO: 24 - protein, 472 aa, Zea mays
MAFYRGLTAVSRLRSRMAQEATTLGGVRWLQMQSASDLDLKSQLQELIPEQQDRLKKLKSEHGKTQLGNITVDMVLGGMRGMTGMLWETSLLDPEEGIRFRGLSIPECQKVLPTAVKGGEPLPEGLLWLLLTGKVPTKEQVDALSKELLARSTVPAHVYKAIDALPVTAHPMTQFTTGVMALQVESEFQKAYDNGLPKSKFWEPTYEDCLNLIARLPPVASYVYRRIFKGGKSIEADNSLDYAANFSHMLGFDDPKMLELMRLYVTIHTDHEGGNVSAHTGHLVGSALSDPYLSFAAALNGLAGPLHGLANQEVLLWIKSVIQETGSDVTTDQLKDYVWKTLKSGKVVPGFGHGVLRKTDPRYSCQREFALKHLPEDPLFQLVSKLYEVVPPILTELGKVKNPWPNVDAHSGVLLNHFGLSEARYYTVLFGVSRSMGIGSQLIWDRALGLPLERPKSVTMEWLENYCKNKAA

SEQ ID NO: 25 - protein, 503 aa, Zea mays
MDRADPARGRLAVLSSHLRGAGAEEAAGLERSPVSAPAPGPRAGALAVVDGRTGKRHEVKVSEDGTVRATDFKKITTGKDDKGLKIYDPGYLNTAPVRSSICYIDGDEGILRYRGYPIEELAESSSFVEVAYLLMYGNLPTQSQLAGWEFAISQHSAVPQGLLDIIQSMPHDAHPMGVLASAMSTLSVFHPDANPALQGQDLYKSKQVRDKQIVRVLGKAPTIAAAAYLRLAGRPPVLPLNTLSYSENFLYMLDSLGDRTYKPNPRLARALDILFILHAEHEMNCSTAAVRHLASSGVDVFTALSGGVGALYGPLHGGANEAVLKMLNEIGSMENIPDFIVGVKNRKRKMSGFGHRVYKNYDPRAKVIRKLADEVFSIVGRDPLIEVAIALEKAALSDEYFIKRKLYPNVDFYSGLIYRAMGFPTEFFPVLFAIPRMGGWLAHWKESLDDPDTKIIRPQQVYTGFWLRHYTPVRERVLSSQSEELGQVATSNATRRRRAGSAL

SEQ ID NO: 26 - DNA, 129 bp, Cucurbita cv. Kurokawa Amakuri
atgcccaccgacatggaattgtcgccttcgaacgttgctcgtcatcgcttggccgttctggcagcgcatctgagcgctgcgtccttggaaccgccggtgatggcttcgtccctcgaggctcattgcgtg

SEQ ID NO: 27 - protein, 43 aa, Cucurbita cv. Kurokawa Amakuri
MPTDMELSPSNVARHRLAVLAAHLSAASLEPPVMASSLEAHCV

SEQ ID NO: 28 - DNA, 81 bp, Citrullus lanatus var. lanatus
atgaaggcctcaattctcagatccgttcgttccgccgtttccagatcctcatcgtcgaatcgcctcttgagccgtagcttt

SEQ ID NO: 29 - protein, 27 aa, Citrullus lanatus var. lanatus
MKASILRSVRSAVSRSSSSNRLLSRSF

SEQ ID NO: 30 - DNA, 99 bp, Artificial
  Corn-codon optimized sequence
atgggatccatgaaagcatccattcttagatcagtccgctcagctgtctcacgctctagctcttctaatagactcctgtcccgtagttttgcaacacat

SEQ ID NO: 31 - protein, 33 aa, Citrullus lanatus var. lanatus
MGSMKASILRSVRSAVSRSSSSNRLLSRSFATH

SEQ ID NO: 32 - DNA, 153 bp, Silene pratensis
atggcttctacactctctaccctctcggtgagcgcatcgttgttgccaaagcaacaaccgatggtcgcctcatcgctaccaaccaacatgggccaagccttgtttggactgaaagccggttctcgtggcagagtgactgcaatggccacatac

SEQ ID NO: 33 - protein, 51 aa, Silene pratensis
MASTLSTLSVSASLLPKQQPMVASSLPTNMGQALFGLKAGSRGRVTAMATY

SEQ ID NO: 34 - DNA, 177 bp, Zea mays
atggcgttccgggtttctggggcggtgctcggtggggccgtaagggctccccgactcaccggcggcggggagggtagtctagtcttccggcacaccggcctcttcttaactcggggtgctcgagttggatgttcggggacgcacggggccatgcgcgcggcggcagcggccaggaag

SEQ ID NO: 35 - protein, 59 aa, Zea mays
MAFRVSGAVLGGAVRAPRLTGGGEGSLVFRHTGLFLTRGARVGCSGTHGAMRAAAAARK

SEQ ID NO: 36 - DNA, 1136 bp, Zea mays
aagcttgctactttctttccttaatgttgatttcccctttgttagatgttctttgtgttatatacactctgtatacaaggatgcgatacacacatcagctagtcctaatgatgccaccgactttacttgaggaaaaggaaacaaatatgatgtggccatcacattctcaataacaatgaccatgtgcgcaatgacataccatcatatttgatatcataaaaataaatttattatcaaagtaaacatatagttcatatatcagatattaaagtgataagaacaaatattacattttatcttatataaaatgacgaaaggtacgagttgaaaaggggtccaaccccttttttatagcttgttcggttgcttgttctccttcggctagcgaggtggtagaatgtgagagtgttgcgcgtggattcccgtcgtagtgttcttaggtgatttctcacggcccatctgtgatatagcgactcattatgtggtgtaatagcccattgggagaaggggagagatatagatctacgtgatttgcgcgtgatgcacgacgaacgaaactggtggtttaaagtagtagaggtttgtcattagtggtgtaagtggtacatatattatccgttcatattcgaatttgatccgtataagggggctaagatctaatccgtatacaagtccaagtattaagtatccgatccatatcggatctttatccgtatccgtatactcaaaatttgatgtttaagattttaatatatatttaaactttataggaactcgataatatttgtatctgatttgaattgtgaaaacaaatatggaacgattaatttcagtctatatccgttccgatatttgtcatgctttgctaaaaatacctttacaaggcatcttgtgcagattatatattaatctgaaatcagttagagaagcctacaaatttgaccaaatgccgagtcatccggcttatcccctttccaactttcagttctgcaagcgccagaaatcgtttttcatctacattgtctttgttgcctgcatacatctataaataggacctgctagatcaatcgcagtccatcggcctcagtcgcacatatctactatactatactctaggaagcaaggacaccaccgcc

SEQ ID NO: 37 - DNA, 1534 bp, Zea mays
ttcccgggcagggagagctatgaggcgtatgtcctcaaagccactttgcattgtgtgaaaccaatatcgatctttgttacttcatcatgcgtgaacatttgtggaaactactagcttacaagcattagtgacagctcagaaaaaagttatctctgaaaggtttcatgtgtaccgtgggaaatgagaaatgttgccaactcaaacaccttcaatatgttgtttgcaggcaaactcttctggaagaaaggtgtctaaaactatgaacgggttacagaaaggtataaaccacggctgtgcattttggaagtatcatctatagatgtctgttgaggggaaagccgtacgccaacgttatttactcagaaacagcttcaacacacagttgtctgctttatgatggcatctccacccaggcacccaccatcacctatctctcgtgcctgtttattttcttgccctttctgatcataaaaaatcattaagagtttgcaaacatgcataggcatatcaataattcaatatgctcatttattaatttgctagcagatcatcttcctactctttactttatttattgtttgaaaaatatgtcctgcacctagggagctcgtatacagtaccaatgcatcttcattaaatgtgaatttcagaaaggaagtaggaacctatgagagtatttttcaaaattaattagcggcttctattatgtttatagcaaaggccaagggcaaaattggaacactaatgatggttggttgcatgagtctgtcgattacttgcaagaaatgtgaacctttgtttctgtgcgtgggcataaaacaaacagcttctagcctcttttacggtacttgcacttgcaagaaatgtgaactccttttcatttctgtatgtggacataatgccaaagcatccaggctttttcatggttgttgatgtctttacacagttcatctccaccagtatgccctcctcatactctatataaacacatcaacagcatcgcaattagccacaagatcacttcgggaggcaagtgcgattttgatcttgcagccacctttttttgttctgttgtaagtatactttcccttaccatctttatctgttagtttaatttgtaattgggaagtattagtggaaagaggatgagatgctatcatctatgtactctgcaaatgcatctgacgttatatgagctgcttcatataatttgaattgctccattcttgccgacaatatattgcaaggtatatgcctagttccatcaaaagttctgttttttcattctaaaagcattttagtggcacacaatttttgtccatgagggaaagggaatctgttttggttactttgcttgaggtgcattcttcatatgtccagttttatggaagtaataaacttcagtttggtcataagatgtcatattaaagggcaaacatatattcaatgttcaattcatcgtaaatgttccctttttgtaaaagattgcatactcatttatttgagttgcaggtgtatctagtagttggaggag

SEQ ID NO: 38 - DNA, 1232 bp, Zea mays
cagcgacctattacacagcccgctcgggcccgcgacgtcgggacacatcttcttcccccttttggtgaagctctgctcgcagctgtccggctccttggacgttcgtgtggcagattcatctgttgtctcgtctcctgtgcttcctgggtagcttgtgtagtggagctgacatggtctgagcaggcttaaaatttgctcgtagacgaggagtaccagcacagcacgttgcggatttctctgcctgtgaagtgcaacgtctaggattgtcacacgccttggtcgcgtcgcgtcgcgtcgcgtcgatgcggtggtgagcagagcagcaacagctgggcggcccaacgttggcttccgtgtcttcgtcgtacgtacgcgcgcgccggggacacgcagcagagagcggagagcgagccgtgcacggggaggtggtgtggaagtggagccgcgcgcccggccgcccgcgcccggtgggcaacccaaaagtacccacgacaagcgaaggcgccaaagcgatccaagctccggaacgcaacagcatgcgtcgcgtcggagagccagccacaagcagccgagaaccgaaccggtgggcgacgcgtcatgggacggacgcgggcgacgcttccaaacgggccacgtacgccggcgtgtgcgtgcgtgcagacgacaagccaaggcgaggcagcccccgatcgggaaagcgttttgggcgcgagcgctggcgtgcgggtcagtcgctggtgcgcagtgccggggggaacgggtatcgtggggggcgcgggcggaggagagcgtggcgagggccgagagcagcgcgcggccgggtcacgcaacgcgccccacgtactgccctccccctccgcgcgcgctagaaataccgaggcctggaccgggggggggccccgtcacatccatccatcgaccgatcgatcgccacagccaacaccacccgccgaggcgacgcgacagccgccaggaggaaggaataaactcactgccagccagtgaagggggagaagtgtactgctccgtcgaccagtgcgcgcaccgcccggcagggctgctcatctcgtcgacgaccaggttctgttccgttccgatccgatccgatcctgtccttgagtttcgtccagatcctggcgcgtatctgcgtgtttgatgatccaggttcttcgaacctaaatctgtccgtgcacacgtcttttctctctctcctacgcagtggattaatcggc

SEQ ID NO: 39 - DNA, 1887 bp, Zea mays
gtttcataaatgcttttcctgattccctcatcaattataaacctatataaggagtttgtggtataagcccgagatttgtccaatacccaaataacttcatctctcccttgagtgggggaatgctctccaagagcatataggagatcaccccaaatctgaaactcactcatagaaagagggcgagtaaaatcaataaaccactcgtcgtccacaaaacagtccgagaccaagacatccttgtccctacttatgtcaaagaaccttgggtacttgattttcaaaggaacatccccgagccatgtatccatccaaaatagggtgcacttcccattttcgatgttgtttattgcgccctatttaaataaatgtttaactttatgtaatccttttccaaaattgggaggtccttttcccattagataagaaaaaaaattgccatcaggcatatacttggctctaacaagtttgtaccaagtattatcagatcattgggagattttccatatccatgtaaccagaaggcactcattcatcggtctgctatctaaaaacaccaaacccccttgctccttcaacctagtcaccatttcccgtttagccataagataacagtttttcttttctgctccttgcgagaagaagtttgctctgatagagttcattttctggtgtgcctctttcgaaagaagatagaagctcatgttatacatcgggaggctactcagactggagttagtcaatatcagcctctccccagaggacagatacttaccctttcatgggtcaagcctcttattcatttttgctagaatagggtccgaaatagcagggttcagatgatggtgagaaatggccacacacctaggttgcggcgtttgtgcgggagctgttggagagggacttgctgatcacgttcggcgagggcaattaaggtatcgtagacatgttctacacggcagccatgtgcgggagcgtggacgtgttcactctactgctcaaccacgccacgaattgccggaacggccaaggcagcgcaaggcgtagtagctccatgttctgtacactgagataatgagtcgagtcgtccacgctgcagcgagaggcgggatcgtggagatgcatagggagttgctcgaggagagacagacgttcaggctattgctcgaccatgcatgtcaccgaggtgttcgacgaattgcaagtcacgctacactgccgactgagcaatacgagctactggcacccagcttgtgatttgaaagatgcacactaacaccaaacagcgaaacacccatgtttcacgctcctcctaccacgtccacgacgaaaactgtatatgtagccacgtccacgtaggaccaaaacgagggacagaggaagcccatgcagcgttttcccgaaagacacgtaaagcagaacgtctccgctccgaggacgacacccgctcacgagcaatccggcagccagccgccgcaccgcagaatcttccccacgccacgctgccactgaaagcgcttcgacctcgtccgtccgttcgctcgctcgcggcgaaccccgcagagcttcccgtgcacgctcgcccgttccgttctgtgtggttggcagcctggcagcaccccacctgtccactcccctccactacgatacgagaccccggatccgtttttgctgtgtgctctaatcaaaaatcaaacaaaccagaagctcctcctcgcctcccatcacttcctacgccacccgcgaagcgcgcccgaggcggcaccccaccgtcgtagtagaagacacgggacgcacccccgcagcctcgctcgctcgctcccctcacttcctccccgcgcgatccacggcccccgccccccgcgctcctgtctgctctccctctccgca

SEQ ID NO: 40 - DNA, 2336 bp, Oryza sativa
gtaaatttacactagcaaaatgcccgtgcttcgctacgggtataatggaaggttaaatgctataaatacacggttaacatgtatgtaataatatttcaagatagataaattgttcattgggttaatgataatcaagactaataagaaacaccattgcatattgatatagtactcatattgtcataacacaagctctctctcaactcttgcatggcacgcgcatgatattacttctaaaatccaacacggattcgcgtggagacgaatcgcttaacagctcatgcgtggacgatagatggtgcggactctaatatgtcgtgttttgcagatgacgaaaagaaaaaaaaattatgtttaattttcaaattaaaaactggttcattataagatttcagaatcactaaatatctgttgaagaaagaggtataaaacttttgacttttcagccatcgaatgtgcatggtctgtgctaatggtggagagaaaaaaaaaggatgtgcatggtccgtgctagtggcggagagaaaaaaattgcacgcaaataggatttgagatatggagacaaagtagacttattccgagtaataataagtgtaaaagttttaggtagggtaggtcaatctggcctaggagaggcccatagacagtgcgagtaataatacaaaactcctaattagtgcatacgcgtcgcgaatttaagtggtgcgcgttttgagcccatccatcgcacagtcgcgtgcgacctattttcctttttttatattatcttttgtttcgagacttctttttcacgcagttggttgccacattgtcttctctattttttcgcacctcttgcgcaaggagtcggataaaaattgaaaaaaaaattcgcacagttggttgctaccgtgtctcctcttttctgttttcgcacctctttttttccttttacgtaattagtttccagtattaccatctactttacttcttctcttttttactaaaaattaaaaattactttaataattaaaaagttataaaaaaataagctataccaaaattgaatactttcacttaagatttcgaaacttcaactcgaatttcgaaaactttcaactacatatttgaaattttcaactattcttttaaaagttttaactaaattatttatgtgttttttctattgaactttgtttttagtgaattccctacctgttattatcctaacttaccgcataaaaaaggaaaaaaaaagataaagcgtcgggaggaatttttttcccaagaaaaaagcgaaaaaaaaacaaataaagaaaaatgaatcaataggtaggagagaaacttaaatgggccaaagcctgtaataataagagtaaaaaggtgagggtggcgggaacgaaccctggtggccacaatgaagattttcttcactaaccaagtaggcaagctgctacttgtgatcacatgttacatcaattaatacatatcctatattaacctagttttgaggtagatccaaaaggatccatcccgactattgagcggattaaaacaattttagcaacatgattctgattcggatgacggacgaaaaaaccatacggaaaccttacgaatttttttattaggtatagatgtcattataaacttttttaaaaaaattcaataatatagtttatgatgtaatatatcacttcacaaacccataacattgcatgatcaaatttaactttctacattttacaaaaaaaataaaaaaaaataattttgaatatacgttaattagttatagtttgcgaaagaacatgtattaacctgattagcgcctcaagatcgtacgttcaaatctccatttgaacgaattttagattgagttatttgagagctaaattttcaatttaaacaattatatatatccggttagatgtacatatcggataaataatacccttttttaaaaaaggattagttgtagtttaatttatttttccattgtgatttataaaaattgaatttgtgaagtgaaatgcaaatggcgaaatgacatttcttcgtttagcgtgaagcaacggagagaaggaacggaagcggtgggacgcgcgcgcacgccacgcgcgtagcgagagcgaaccagctcatccaccccgcgatccgtttttgctgtgccccaccgcctcagcgcctctccctcgcttaaaaccaaacccacacacccacctcttctctctctctcatcgtctccgcgactcagcccactcctctctctccaccaccaccaccaccaccaccaccgcccgccaagcgcggcccgcccgacacagcagcagcaggatcggcggagaggaggggggatc

SEQ ID NO: 41 - DNA, 253 bp, Agrobacterium tumefaciens T-DNA
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

(19) United States
(12) Patent Application Publication
(10) Pub. No.: US 2012/0216315 A1
(43) Pub. Date: Aug. 23, 2012

(54) Methods in Increasing Grain Value by Improving Grain Yield and Quality

(75) Inventors: Hanping Guan, Chapel Hill, NC (US); Beomseok Seo, Morrisville, NC (US)

(73) Assignee: BASF Plant Science, Ludwigshafen (DE)

(21) Appl. No.: 12/996,020

(22) PCT Filed: Jun. 10, 2009
(86) PCT No.: PCT/EP09/57151

§ 371 Date: Dec. 3, 2010

Related U.S. Application Data
(60) Provisional application No. 61/061,231, filed on Jun. 13, 2008.

(51) Int. Cl. A01H 5/00; A01H 1/06; A01H 5/10; C12N 15/82

(52) U.S. Cl. 800/281; 800/298; 435/320.1; 800/320.1; 800/320.3; 800/320.2; 800/320; 800/312; 800/318; 800/317.4; 800/317.2; 800/314; 800/317.3; 800/317.1; 800/306; 800/305; 800/278

(57) ABSTRACT

The invention provides a transgenic plant, which expresses a transgene encoding a citrate synthase (CS) wherein the transgenic plant seed of the invention is characterized by increased yield and/or enhanced levels of protein, essential amino acids or oil, when compared to an isoline that does not express the transgene; and also provides methods of producing transgenic plants with economically relevant traits and provides expression vectors comprising polynucleotides encoding Citrate Synthase.

### BACKGROUND OF THE INVENTION

1. Field of the Invention

The present invention is directed to transgenic plants, expressing the transgene citrate synthase (CS) and the methods of use. The transgenic plants that express transgene CS, particularly when expressed in seeds or in seeds and further targeted to the cell compartments such as plastids, have higher levels of grain yield, and/or amino acids, in particular cysteine, and/or oil when compared to isoline controls which do not contain the transgene linked to a seed preferred promoter or further operably linked to a cell compartment targeting sequence.

2. Background Art

Cereal grain is one of the most important renewable energy sources for humans and animals. With increasing world population and limited arable land, the demand for food, feed, fiber and biofuels are increasing. It is essential and invaluable to increase grain yield per acre and enhance grain nutritional value per acre to meet these demands. Since over 90% of corn grain is used for animal feed and ethanol production today, corn is one of the most important crops for animal nutrition. Grain of yellow dent corn consists of 60-70% starch, 8-10% protein, and 3-4% oil. However, despite these valuable feed components, yellow dent corn does not contain sufficient calories and essential amino acids to support optimal growth and development in most animals. Therefore, to compensate for these shortcomings, it is necessary to supplement yellow dent corn-based feed with other nutrients. Most commonly, yellow dent corn is mixed with soybean meal to improve the amino acid composition of the feed. Unfortunately, animals lack the enzymes necessary to digest the non-starch based polysaccharides present in soybean meal, and corn and soybean feed mixtures result in high manure volume. In addition, soybean meal is expensive. Furthermore, to improve caloric content, corn-based animal feed is also supplemented with fats, such as animal offal and feed-grade animal and vegetable fats, which may include by-products of the restaurant, soap, and refinery industries. Use of animal offal to supplement cattle feed has been discontinued because of its association with bovine spongiform encephalopathy and Creutzfeldt-Jakob disease. Improvements to grain yield and the nutritional qualities of corn grain will increase value per acre, energy per acre, and improve feed efficiency and reduce environmental impact and other costs associated with meat production.

Respiration, including the tricarboxylic acid (TCA) cycle, not only provides the energy for synthesizing the storage compounds but also generates intermediates for oil and amino acid biosyntheses. Citrate synthase (CS) catalyzes the formation of citrate from oxyloacetate and acetyl CoA. This is the first committed step in the TCA cycle, which is normally present in the mitochondrion. CS plays an important role in the TCA cycle and metabolism. Attempts have been made to engineer citrate synthase to improve crop productivity. US2005/0137386 describes a process for obtaining transgenic plants which have improved capacity for the uptake of nutrients and tolerance to toxic compounds that are present in the soil. Research done by de la Fuente et. al. showed that expression of a Pseudomonas aeruginosa citrate synthase gene in tobacco increased aluminum tolerance (Science 276: 1566-1568, 1997). Lopez et. al. reported enhanced phosphorus uptake due to organic acids solubilizing poorly-soluble forms of phosphate (Nature Biotech 18: 450-453, 2000). However, this approach appears to be subject to environmental influences as another group was unable to reproduce these findings using these same plants as well as ones engineered to express the citrate synthase gene to a higher level (Delhaize et al. Plant Physiology 125: 2059-2067, 2001).

WO 2004/056968 disclosed that over-expression of the Arabidopsis citrate synthase gene (At3g58750) conferred as much as a 7% increase in seed oil compared to nontransgenic control when measured by Near Infrared Spectroscopy. US Patent Application Publication Nos 2003/0233670 and 2005/0108791 disclosed citrate synthases from Xyllela fastidia, E. coli, rice, maize, and soybean and their use in improving phosphate uptake of transgenic plants. Over-expression of both mitochondrial and cytoplasmic forms of citrate synthase has been reported to improve phosphate uptake in model plants (Lopez-Bucio et al., 2000; Kayama et al., 2000). However, there are reports that expression of a Pseudomonas aeruginosa citrate synthase gene in tobacco is not associated with either enhanced citrate accumulation or efflux (Plant Physiology, 2001, Vol. 125:2059-2067). The authors suggest that expression of CS in plants is unlikely to be a robust and easily reproducible strategy for enhancing the Aluminum tolerance and P-nutrition of crops.

While the bound amino acids (protein composition) account for 90-99% of total amino acids in corn seed, free amino acids account for 1-10% of the total amino acids. There are serious challenges to further increase essential amino acid contents. One challenge is that increasing free amino acid concentration does not always result in total amino acid increase because the flux and incorporation of free amino acid into protein may become limiting. Secondly, accumulation of free amino acids is often associated with adverse agronomic performance, such as stunted growth, therefore affecting marketability. From the nutritional quality perspective, an ideal grain would be one with improved contents of oil, protein, and essential amino acids such as valine, threonine, cysteine, methionine, lysine and/or arginine.

A need continues to exist for increased grain yield and for plant grain that has desirable agronomic characteristics and with increased levels of essential amino acids, protein or oil.

### SUMMARY OF THE INVENTION

The present invention provides a transgenic plant, and its parts, expressing a gene encoding the citrate synthase (CS) protein in the transgenic plant seed, or in the intracellular compartment in the seed, wherein the CS confers higher levels of grain yield and/or higher levels of amino acids (such as cysteine, methionine, arginine, threonine, lysine and/or valine) and/or oil when compared to an isoline plant or seed that does not express the transgenic citrate synthase protein in this manner. The present invention also includes methods of using the polynucleotides and vectors described herein to confer economically relevant traits to the resulting transgenic plants and its parts.

In one embodiment, the invention provides a transgenic plant, and its parts, comprising a polynucleotide encoding a heterologous citrate synthase, expressed in the seed or in an intracellular cell compartment of the seed, wherein the polynucleotide is selected from the group consisting of: a) a polynucleotide having a sequence as defined in SEQ ID NO:1, 2, 3, 4, 5, 6, 7, 8, 12, 13, 14, or 15; b) a polynucleotide encoding a polypeptide having a sequence as defined in SEQ ID NO:16, 17, 18, 19, 22, 23, 24, or 25; c) a polynucleotide having at least 70% sequence identity to a polynucleotide having a sequence as defined in SEQ ID NO:1, 2, 3, 4, 5, 6, 7, 8, 12, 13, 14, or 15; d) a polynucleotide encoding a polypeptide having at least 70% sequence identity to a polypeptide having a sequence as defined in SEQ ID NO:16, 17, 18, 19, 22, 23, 24, or 25; e) a polynucleotide hybridizing under stringent conditions to a polynucleotide having a sequence as defined in SEQ ID NO:1, 2, 3, 4, 5, 6, 7, 8, 13, 14, or 15; f) a polynucleotide hybridizing under stringent conditions to a polynucleotide encoding a polypeptide having a sequence as defined in SEQ ID NO:16, 17, 18, 19, 22, 23, 24, or 25; and g) a polynucleotide complementary to any of the polynucleotides of a) through f). Additional embodiments of the aforementioned transgenic plant provide that the plant is a monocot or a dicot or, more specifically, the plant is selected from the group consisting of maize, wheat, rice, barley, oat, rye, sorghum, banana, ryegrass, pea, alfalfa, soybean, carrot, celery, tomato, potato, cotton, tobacco, pepper, oilseed rape, beet, cabbage, cauliflower, broccoli, lettuce and Arabidopsis thaliana. A further embodiment of the previously described transgenic plant provides, wherein expression of the polynucleotide is capable of conferring to the plant an economically relevant trait and further wherein the economically relevant trait is selected from the group consisting of: at least 2% increase in oil content over the oil content of an isoline, at least 4% increase in cycteine of the cysteine content of an isoline, and at least 3 bushel per acre yield increase over bushel per acre yield of an isoline. Another embodiment of the previously described transgenic plant provides, wherein the plant has an increase of about 3-19 bushels per acre in grain yield over the grain yield of an isoline.

Another embodiment provides for seed of the previously described transgenic plant, wherein (a) the seed has an increase of at least 3% in one or more amino acids selected from the group consisting of: threonine, cysteine, valine, methionine, lysine, and arginine, over the amounts of said amino acid in an isoline; or (b) the seed has an increase of about 4%-27% in cysteine content over the cysteine content of an isoline; or (c) the seed has an increase of about 2%-13% in methionine content over the methionine content of an isoline; or (d) the seed has an increase of about 2%-10% in oil content over the oil content of an isoline. Further embodiments provide a seed produced from the aforementioned transgenic plant, wherein the seed comprises the polynucleotide and a further embodiment where expression of the polynucleotide in the seed confers an economically relevant trait to the seed that is not present at the same level in an isoline.

In another embodiment, the invention provides a transgenic plant seed expressing a CS gene in said seed, wherein said seed comprises an economically relevant trait of agronomic or nutritional importance, selected from the group consisting of:
a) an increase of at least 3 bushels per acre in grain yield over the isoline;
b) an increase of at least 3 bushels per acre in grain yield over the isoline and the seed has at least 4% more cysteine than the isoline seed;
c) at least 3 bushels/acre increase in grain yield over the isoline and the seed has a at least 4% increase of cysteine and at least 2% increase in methionine than the isoline seed; and
d) at least 3 bushels per acre increase in grain yield over the isoline and the seed has at least 4% more cysteine and at least 2% more oil than the isoline seed.

Another embodiment of the invention relates to a method of producing a transgenic plant having an economically relevant trait, wherein the method comprises the steps of: A) introducing into the plant an expression vector comprising a seed-preferred transcription regulatory element operably linked to a polynucleotide, wherein the polynucleotide encodes a polypeptide that is capable of conferring the economically relevant trait, and wherein the polynucleotide is selected from the group consisting of:
a) a polynucleotide having a sequence as defined in SEQ ID NO:1, 2, 3, 4, 5, 6, 7, 8, 12, 13, 14, or 15;
b) a polynucleotide encoding a polypeptide having a sequence as defined in SEQ ID NO:16, 17, 18, 19, 22, 23, 24, or 25;
c) a polynucleotide having at least 70% sequence identity to a polynucleotide having a sequence as defined in SEQ ID NO:1, 2, 3, 4, 5, 6, 7, 8, 12, 13, 14, or 15;
d) a polynucleotide encoding a polypeptide having at least 70% sequence identity to a polypeptide having a sequence as defined in SEQ ID NO:16, 17, 18, 19, 22, 23, 24, or 25;
e) a polynucleotide hybridizing under stringent conditions to a polynucleotide having a sequence as defined in SEQ ID NO:1, 2, 3, 4, 5, 6, 7, 8, 12, 13, 14, or 15;
f) a polynucleotide hybridizing under stringent conditions to a polynucleotide encoding a polypeptide having a sequence as defined in SEQ ID NO:16, 17, 18, 19, 22, 23, 24, or 25; and
g) a polynucleotide complementary to any of the polynucleotides of a) through f) and B) selecting transgenic plants with the economically relevant trait.

Another embodiment of the invention provides a transgenic plant, and its parts, over-expressing an active heterologous citrate synthase in the cytosol of a seed, wherein the isolated CS protein is encoded by polynucleotide selected from the group consisting of:
a) a polynucleotide having a sequence as defined in SEQ ID NO:1, 2, 3, 4, 5, 6, 7, 8, 12, 13, 14, or 15;
b) a polynucleotide encoding a polypeptide having a sequence as defined in SEQ ID NO:16, 17, 18, 19, 22, 23, 24, or 25;
c) a polynucleotide having at least 70% sequence identity to a polynucleotide having a sequence as defined in SEQ ID NO:1, 2, 3, 4, 5, 6, 7, 8, 12, 13, 14, or 15;
d) a polynucleotide encoding a polypeptide having at least 70% sequence identity to a polypeptide having a sequence as defined in SEQ ID NO:16, 17, 18, 19, 22, 23, 24, or 25;
e) a polynucleotide hybridizing under stringent conditions to a polynucleotide having a sequence as defined in SEQ ID NO:1, 2, 3, 4, 5, 6, 7, 8, 12, 13, 14, or 15;
f) a polynucleotide hybridizing under stringent conditions to a polynucleotide encoding a polypeptide having a sequence as defined in SEQ ID NO:16, 17, 18, 19, 22, 23, 24, or 25; and
g) a polynucleotide complementary to any of the polynucleotides of a) through f).

A further embodiment of the present invention provides for an expression vector comprising a seed-preferred transcription regulatory element operably linked to a polynucleotide, wherein the polynucleotide is selected from the group consisting of:
a) a polynucleotide having a sequence as defined in SEQ ID NO:1, 2, 3, 4, 5, 6, 7, 8, 12, 13, 14, or 15;
b) a polynucleotide encoding a polypeptide having a sequence as defined in SEQ ID NO:16, 17, 18, 19, 22, 23, 24, or 25;
c) a polynucleotide having 70% sequence identity to a polynucleotide having a sequence as defined in SEQ ID NO:1, 2, 3, 4, 5, 6, 7, 8, 12, 13, 14, or 15.;
d) a polynucleotide encoding a polypeptide having at least 70% sequence identity to a polypeptide having a sequence as defined in SEQ ID NO:16, 17, 18, 19, 22, 23, 24, or 25;
e) a polynucleotide hybridizing under stringent conditions to a polynucleotide having a sequence as defined in SEQ ID NO:1, 2, 3, 4, 5, 6, 7, 8, 12, 13, 14, or 15;
f) a polynucleotide hybridizing under stringent conditions to a polynucleotide encoding a polypeptide having a sequence as defined in SEQ ID NO:16, 17, 18, 19, 22, 23, 24, or 25; and
g) a polynucleotide complementary to any of the polynucleotides of a) through f).

The expression vector may further be operably linked to an intracellular targeting sequence. Also, the expression vector's seed-preferred transcription regulatory element may be an endosperm-preferred promoter. The inventors determined that targeting the expression of an active heterologous CS in plastid or cytosol of seeds is effective in increasing grain yield and/or increasing grain nutrient content such as the essential amino acid cysteine.

Another embodiment of the invention relates to a method of producing a transgenic plant having an economically relevant trait, wherein the method comprises the steps of: A) introducing into the plant an expression vector comprising the polynucleotide of the invention as described above, wherein expression of the polynucleotide confers the economically relevant trait to the plant; and B) selecting transgenic plants with the economically relevant trait. In one embodiment, the economically relevant trait of a transgenic plant is selected from the group consisting of:
a) an increase of at least 3 bushels per acre in grain yield over the isoline;
b) an increase of at least 3 bushels per acre in grain yield over the isoline and the seed has at least 4% more cysteine than the isoline seed;
c) at least 3 bushels/acre increase in grain yield over the isoline and the seed has a at least 4% increase of cysteine and at least 2% increase in methionine than the isoline seed; and
d) at least 3 bushels per acre increase in grain yield over the isoline and the seed has at least 4% more cysteine and at least 2% more oil than the isoline seed.

Another embodiment of the invention relates to a method of producing a transgenic plant having an economically relevant trait, wherein the method comprises the steps of: A) introducing into the plant an expression vector comprising the polynucleotide of the invention as described above, wherein expression of the polynucleotide confers the economically relevant trait to the plant; and B) selecting transgenic plants with the economically relevant trait. In one embodiment, the economically relevant trait of a transgenic plant is selected from the group consisting of:
a) an increase of about 3-19 bushels per acre in grain yield over the isoline;
b) an increase of about 3-19 bushels per acre in grain yield over the isoline and the seed has about 4-27% more cysteine than the isoline seed;
c) an increase of about 3-19 bushels/acre in grain yield over the isoline and the seed has about 4-27% increase of cysteine and about 2-18% increase in methionine than the isoline seed; and
d) an increase of about 3-19 bushels per acre in grain yield over the isoline and the seed has about 4-27% more cysteine and about 2-7% more oil than the isoline seed.

Another embodiment of the invention relates to a method of producing a transgenic plant having an economically relevant trait, wherein the method comprises the steps of: A) introducing into the plant an expression vector comprising the polynucleotide of the invention as described above, wherein expression of the polynucleotide confers the economically relevant trait to the plant; and B) selecting transgenic plants with the economically relevant trait. In one embodiment, the economically relevant trait of a transgenic plant is selected from the group consisting of:
a) an increase of about 3-10 bushels per acre in grain yield over the isoline;
b) an increase of about 3-10 bushels per acre in grain yield over the isoline and the seed has about 4-15% more cysteine than the isoline seed;
c) an increase of about 3-10 bushels/acre in grain yield over the isoline and the seed has about 4-15% increase of cysteine and about 2-10% increase in methionine than the isoline seed; and
d) an increase of about 3-10 bushels per acre in grain yield over the isoline and the seed has about 4-15% more cysteine and about 2-5% more oil than the isoline seed.

Another embodiment of the invention relates to a method of producing a transgenic plant having an economically relevant trait, wherein the method comprises the steps of: A) introducing into the plant an expression vector comprising the polynucleotide of the invention as described above, wherein expression of the polynucleotide confers the economically relevant trait to the plant; and B) selecting transgenic plants with the economically relevant trait. In one embodiment, the economically relevant trait of a transgenic plant is selected from the group consisting of:
a) at least 2% increase in oil content over the oil content of an isoline;
b) at least 4% increase in cysteine of the cysteine content of an isoline;
c) an increase of about 4%-27% in cysteine content over the cysteine content of an isoline;
d) an increase of at least about 3% in one or more amino acids selected from the group consisting of: threonine, cysteine, valine, methionine, lysine, and arginine, over the amounts of said amino acid in an isoline; and
e) an increase of about 2-10% in oil content in seeds over the oil content in seeds of isoline.

Another embodiment of the present invention is a transgenic plant and its parts produced by any of the previously described methods.

### BRIEF DESCRIPTION OF THE DRAWINGS

FIG. 1a-b shows the genes and elements along with corresponding SEQ ID NOs.

FIG. 2 shows the protein sequence global identity/similarity percentages of AnaCS (SEQ ID NO:19), E. coliCS1 (SEQ ID NO:16), MaizeCS1 (SEQ ID NO:24), MaizeCS2 (SEQ ID NO:25), PumpkinCS (SEQ ID NO:20), RiceCS1 (SEQ ID NO:22), RiceCS2 (SEQ ID NO:23), YeastCS1 (SEQ ID NO:17), and YeastCS2 (SEQ ID NO:18). The sequence analysis was performed in Vector NTI9 software suite (gap opening penalty=10, gap extension penalty=0.05, gap separation penalty=8).

FIG. 3 shows the protein sequence local identity/similarity percentages of AnaCS (SEQ ID NO:19), E. coliCS1 (SEQ ID NO:16), MaizeCS1 (SEQ ID NO:24), MaizeCS2 (SEQ ID NO:25), PumpkinCS (SEQ ID NO:20), RiceCS1 (SEQ ID NO:22), RiceCS2 (SEQ ID NO:23), YeastCS1 (SEQ ID NO:17), and YeastCS2 (SEQ ID NO:18). The sequence analysis was performed in Vector NTI9 software suite (gap opening penalty=10, gap extension penalty=0.05, gap separation penalty=8).

FIG. 4 shows the DNA sequence global identity percentage of AnaCS (SEQ ID NO:7), E. coliCS1 (SEQ ID NO:1), MaizeCS1 (SEQ ID NO:14), MaizeCS2 (SEQ ID NO:15), PumpkinCS (SEQ ID NO:9), RiceCS1 (SEQ ID NO:12), RiceCS2 (SEQ ID NO:13), YeastCS1 (SEQ ID NO:3), and YeastCS2 (SEQ ID NO:5). The DNA analysis was performed in Vector NTI9 software suite (gap opening penalty=10, gap extension penalty=0.05, gap separation penalty=8).

FIG. 5 displays the phylogenetic relationships of the proteins: Anabaena_CS (SEQ ID NO:19), E. coli_CS1 (SEQ ID NO:16), Maize_CS1 (SEQ ID NO:24), Maize_CS2 (SEQ ID NO:25), Pumpkin_CS (SEQ ID NO:20), Rice_CS1 (SEQ ID NO:22), Rice_CS2 (SEQ ID NO:23), Yeast_CS1 (SEQ ID NO:17), and Yeast_CS2 (SEQ ID NO:18). The sequence analysis was performed in Vector NTI9 software suite (gap opening penalty=10, gap extension penalty=0.05, gap separation penalty=8).

FIG. 6a-c show the protein sequence alignment of Anabaena_CS (SEQ ID NO:19), E. coli_CS1 (SEQ ID NO:16), Maize_CS1 (SEQ ID NO:24), Maize_CS2 (SEQ ID NO:25), Pumpkin_CS (SEQ ID NO:20), Rice_CS1 (SEQ ID NO:22), Rice_CS2 (SEQ ID NO:23), Yeast_CS1 (SEQ ID NO:17), and Yeast_CS2 (SEQ ID NO:18). The sequence analysis was performed in Vector NTI9 software suite (gap opening penalty=10, gap extension penalty=0.05, gap separation penalty=8). Identical and conservative amino acids are denoted by uppercase letters in bold while similar amino acids are denoted by lowercase letters.

FIG. 7 shows the protein sequence alignment of Maize_CS2 (SEQ ID NO:25), Pumpkin_CS (SEQ ID NO:20), and Rice_CS2 (SEQ ID NO:23). The sequence analysis was performed in Vector NTI9 software suite (gap opening penalty=10, gap extension penalty=0.05, gap separation penalty=8). Identical and conservative amino acids are denoted by uppercase letters in bold while similar amino acids are denoted by lowercase letters.

FIG. 8 shows the protein sequence alignment of: Maize_CS1 (SEQ ID NO:24), Pumpkin_CS (SEQ ID NO:20), Rice_CS1 (SEQ ID NO:22), Yeast_CS1 (SEQ ID NO:17), and Yeast_CS2 (SEQ ID NO:18). The sequence analysis was performed in Vector NTI9 software suite (gap opening penalty=10, gap extension penalty=0.05, gap separation penalty=8). Identical and conservative amino acids are denoted by uppercase letters in bold while similar amino acids are denoted by lowercase letters.

FIG. 9 shows the protein sequence alignment of Anabaena_CS (SEQ ID NO:19) and E. coli_CS1 (SEQ ID NO:16). The sequence analysis was performed in Vector NTI9 software suite (gap opening penalty=10, gap extension penalty=0.05, gap separation penalty=8). Identical and conservative amino acids are denoted by uppercase letters in bold while similar amino acids are denoted by lowercase letters.

FIG. 10a shows the activity of yeast CS2 (construct CS1008) in maize developing seeds (23DAP). The closed squares denote the native CS activity from isoline control corn seed and the open squares denote maize CS peak and an additional activity peak of yeast CS2 around fraction 29. FIG. 10b shows the activity of yeast CS1 (construct CS1012) in maize developing seeds (23DAP). The closed squares denote the native CS activity from isoline control corn seed and the open squares denote the maize native CS peak and an additional activity peak of yeast CS1 around fraction 25. Following the same pattern of closed squares denoting the native maize CS peak in the non-transformed isoline and open squares denoting both the native Maize CS peak and the additional activity peak of the transgenic CS in maize developing seeds (23 DAP); FIG. 10c shows an activity peak of Yeast CS1 (CS1001) at about fraction 25, FIG. 10d shows an activity peak of E. coli CS1 (CS 1002) at about fraction 33, FIG. 10e shows an activity peak of E. coli CS1 (CS1004) at about fraction 32, FIG. 10f shows an activity peak of Anabaena CS (CS1005) at about fraction 30, FIG. 10g shows an activity peak of Anabaena CS (CS 1007) at about fraction 30.

FIG. 11 shows the effect of expressing CS in various constructs comprising heterologous CS on grain nutrient composition in T2 seeds.

FIG. 12 shows the effect (average of all events tested across 3-6 locations) of expressing heterologous CS in a corn hybrid (produced by crossing event with the proprietary inbred B) on grain yield and composition, in particular when operably linked to a seed preferred promoter or operably linked to a seed preferred promoter and an intracellular targeting sequence.

FIG. 13 shows the effect of expressing heterologous CS in a corn hybrid (produced by crossing event with the proprietary inbred B) in an individual event (two events selected from a construct that were tested for grain yield (6 locations) and composition (F2 grain from 3 locations), in particular when operably linked to a seed preferred promoter or operably linked to a seed preferred promoter and an intracellular targeting sequence.

FIG. 14 shows the effect of expressing heterologous CS (E. coli CS1 and Yeast CS2) in three corn hybrids (produced by crossing event with the proprietary inbreds A, B and C, individually). Grain yield were tested in 12 locations across 4 Midwest states. Nutrient composition testing of F2 grain was conducted in 3 locations.

FIG. 15 shows the effect of expressing heterologous CS (Yeast CS1 with different promoters and intracellular targeting) in three corn hybrids (produced by crossing event with the proprietary inbreds A, B and C, individually). Grain yield were tested in 12 locations across 4 Midwest states. Nutrient composition testing of F2 grain was conducted in 3 locations.

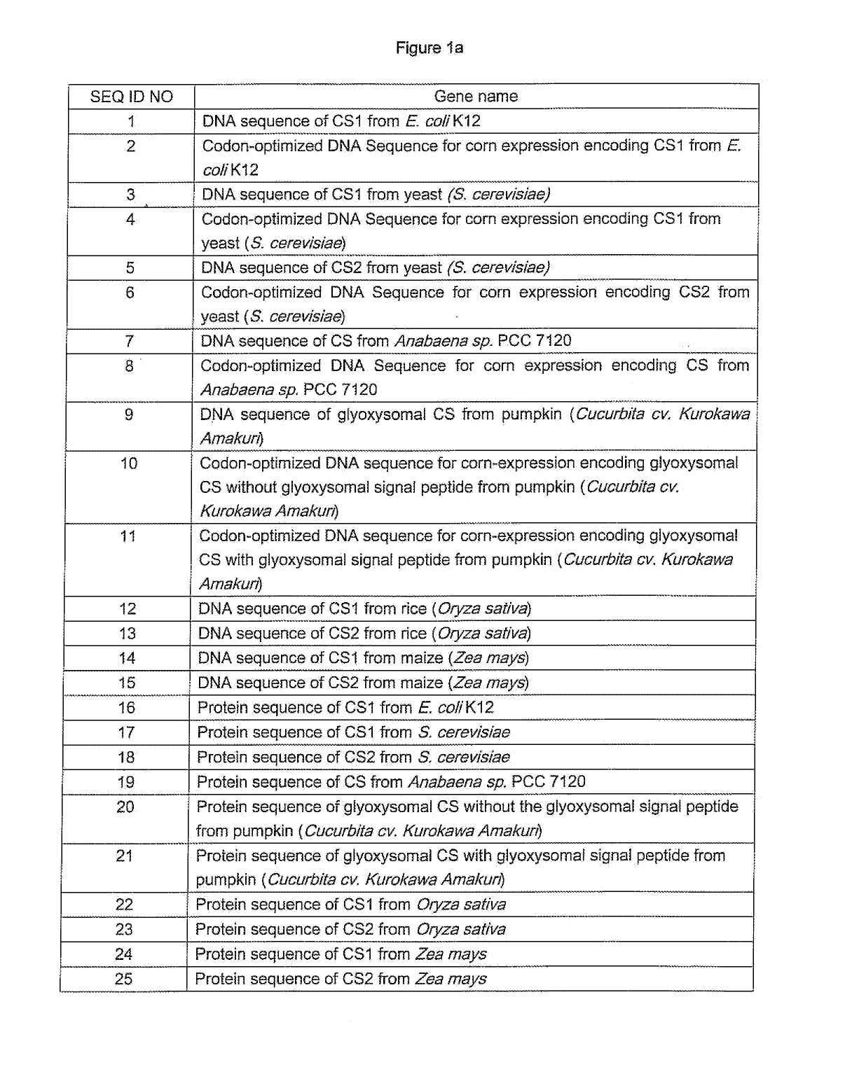

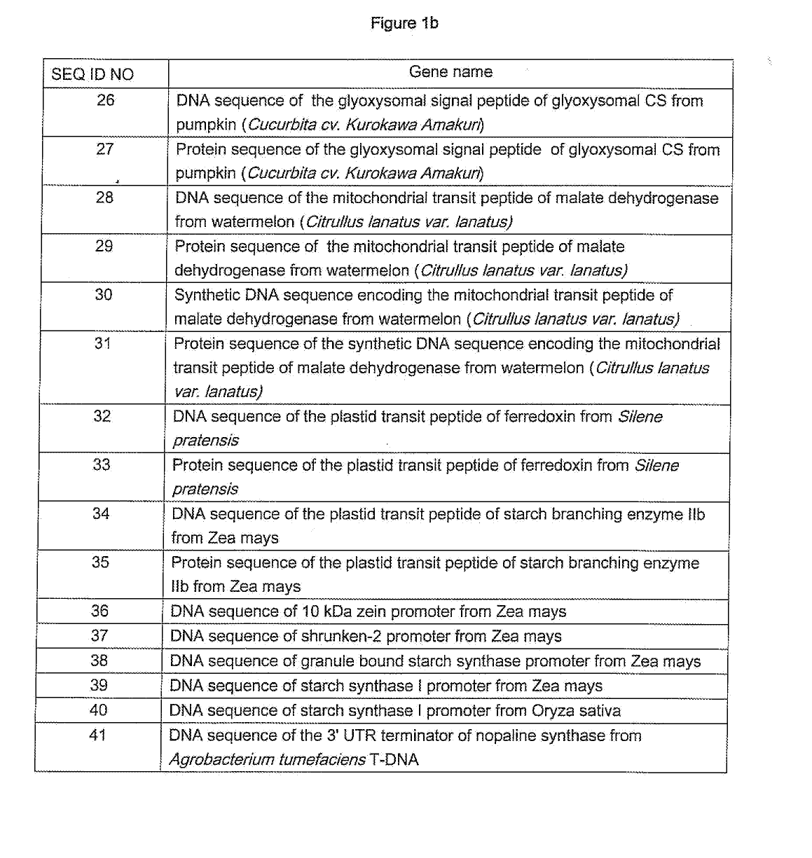

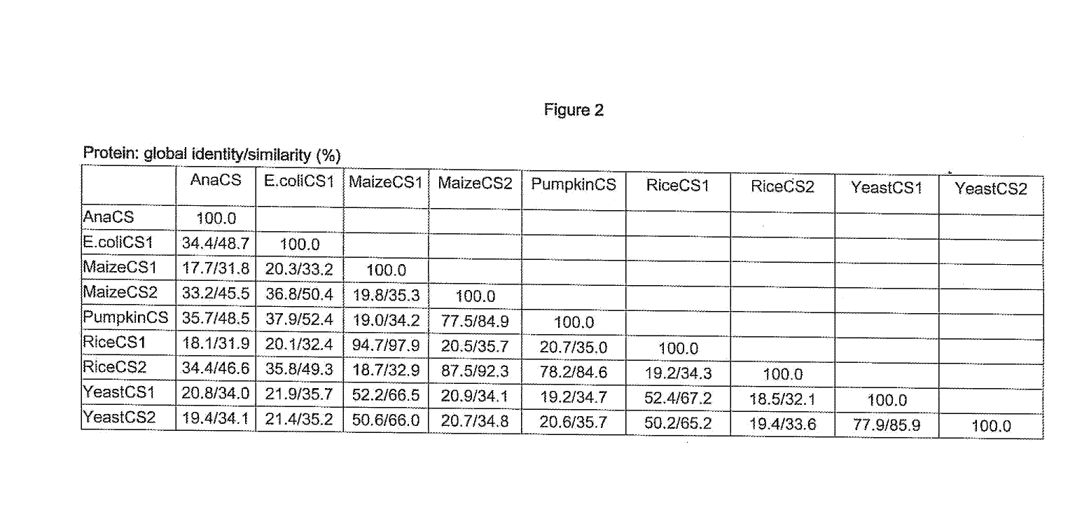

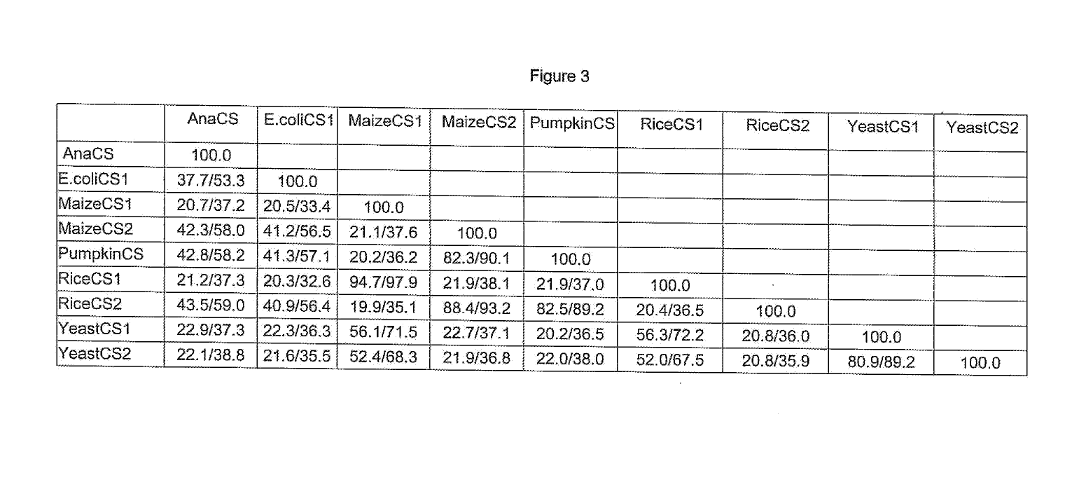

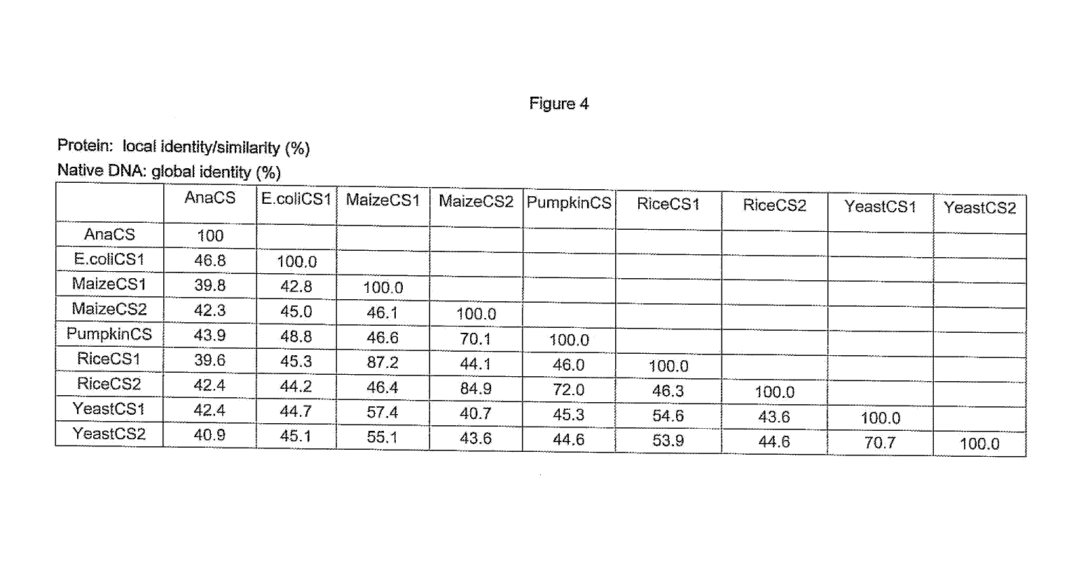

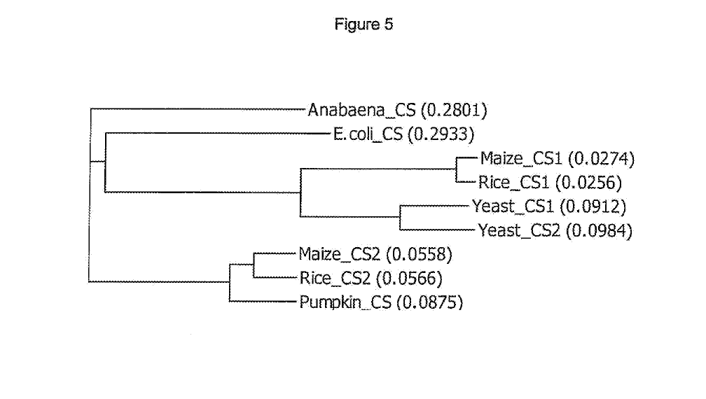

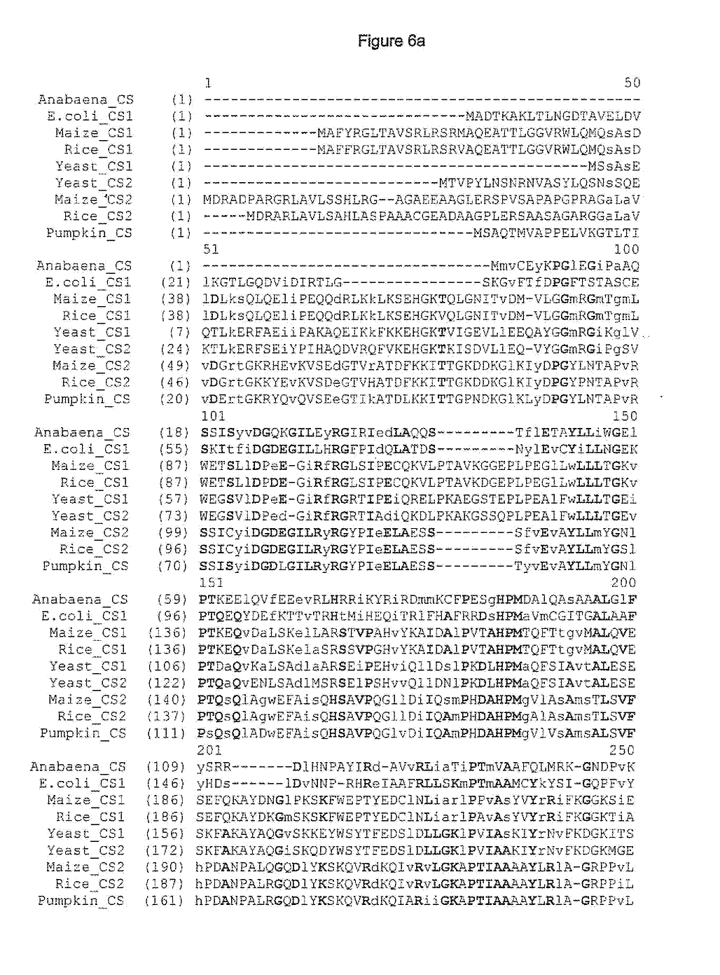

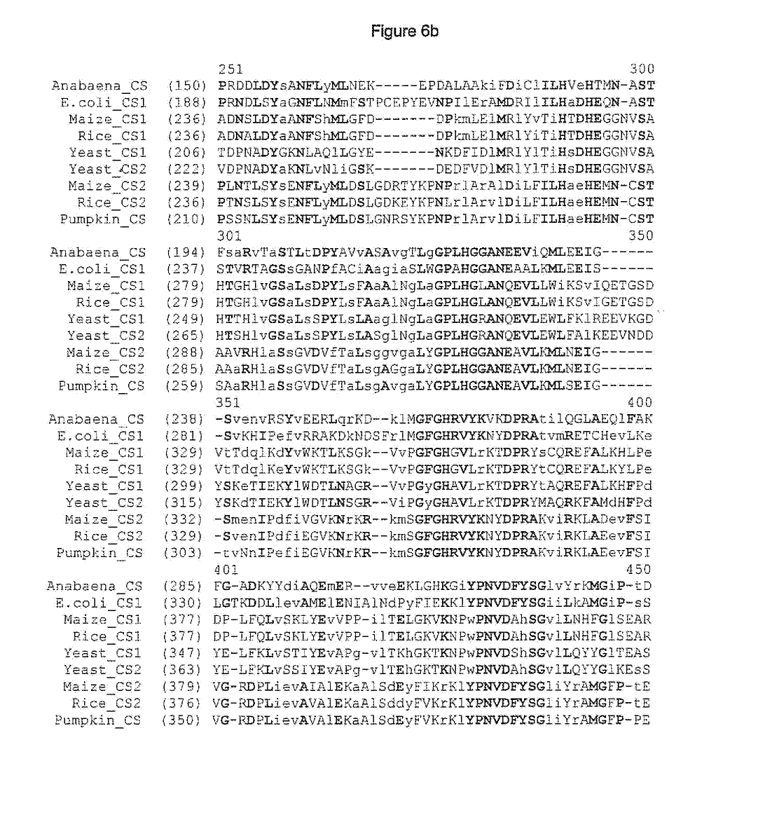

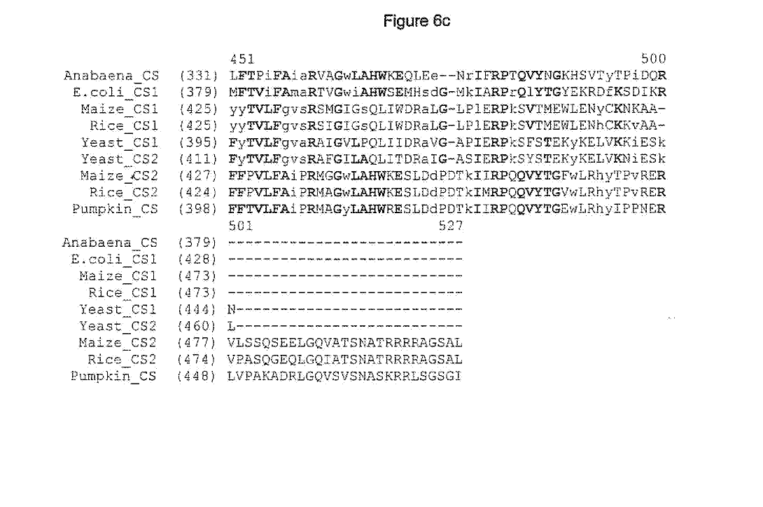

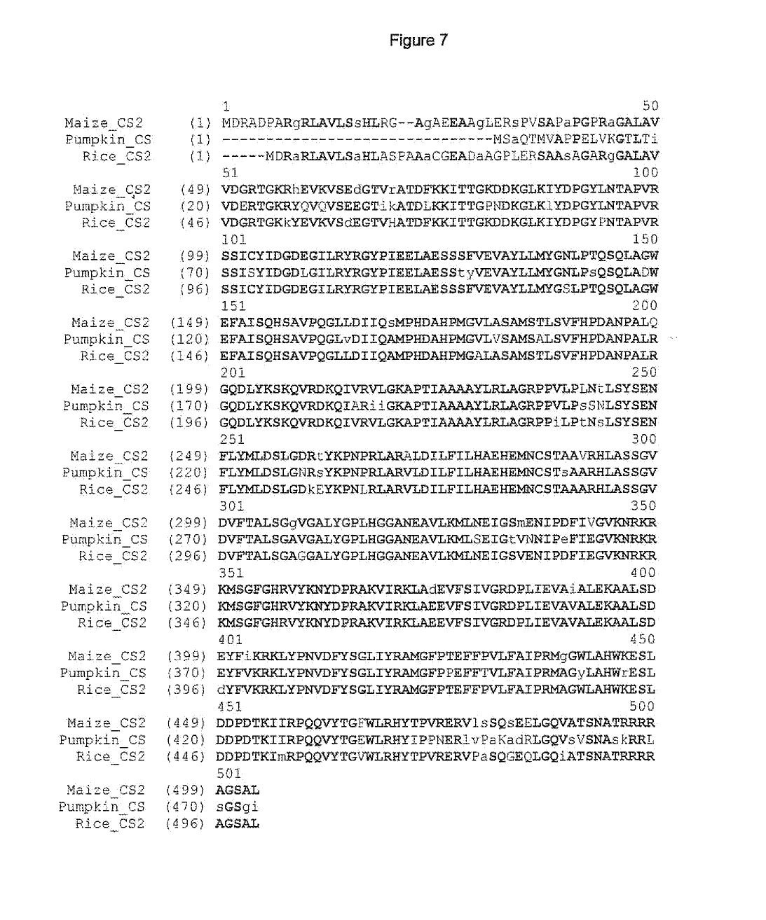

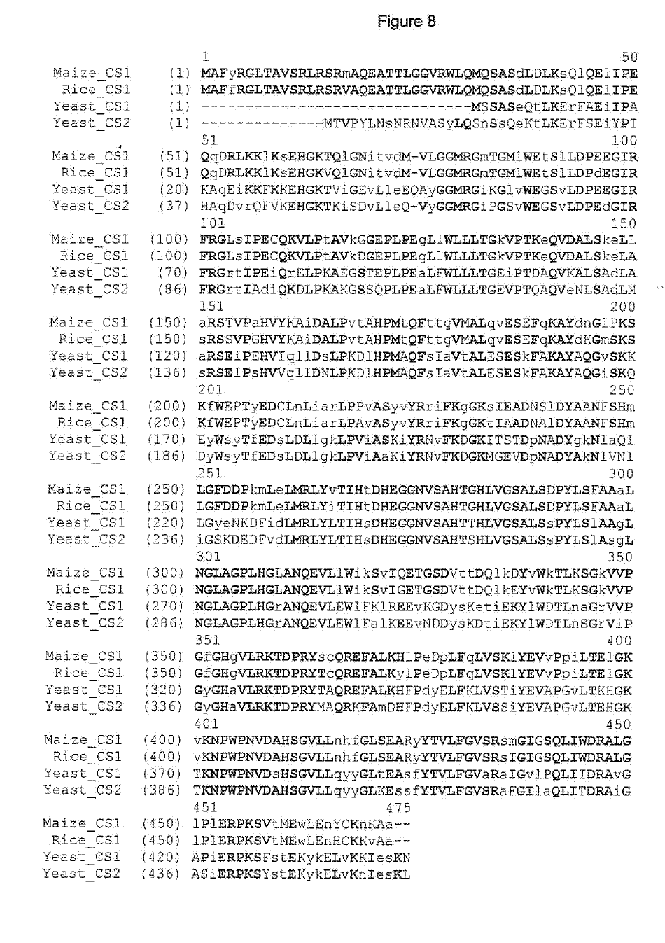

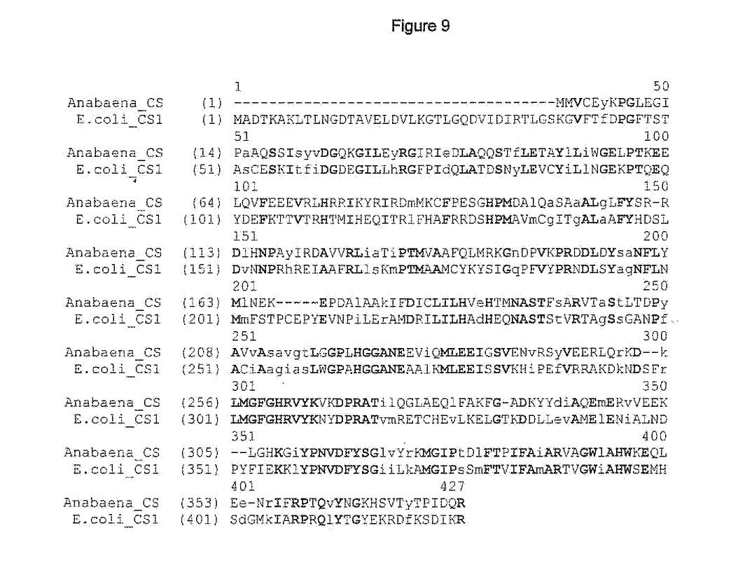

### DETAILED DESCRIPTION OF THE INVENTION

The present invention may be understood more readily by reference to the following detailed description of the embodiments of the invention and the examples included herein. Unless otherwise noted, the terms used herein are to be understood according to conventional usage by those of ordinary skill in the relevant art. In addition to the definitions of terms provided below, definitions of common terms in molecular biology may also be found in Rieger et al., 1991 Glossary of Genetics: Classical and Molecular, 5th Ed., Berlin: Springer-Verlag; and in Current Protocols in Molecular Biology, F. M. Ausubel et al., Eds., Current Protocols, a joint venture between Greene Publishing Associates, Inc. and John Wiley & Sons, Inc. (1998 Supplement).

Throughout this application, various publications are referenced. The disclosures of all of these publications and those references cited within those publications in their entireties are hereby incorporated by reference into this application in order to more fully describe the state of the art to which this invention pertains. This application claims priority to U.S. Provisional Patent application 60/061,231, hereby incorporated by reference into this application. Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook and Russell, 2001 Molecular Cloning, Third Edition, Cold Spring Harbor, Plainview, N.Y.; Sambrook et al., 1989 Molecular Cloning, Second Edition, Cold Spring Harbor Laboratory, Plainview, N.Y.; Maniatis et al., 1982 Molecular Cloning, Cold Spring Harbor Laboratory, Plainview, N.Y.; Wu (Ed.) 1993 Meth. Enzymol. 218, Part I; Wu (Ed.) 1979 Meth Enzymol. 68; Wu et al., (Eds.) 1983 Meth. Enzymol. 100 and 101; Grossman and Moldave (Eds.) 1980 Meth. Enzymol. 65; Miller (Ed.) 1972 Experiments in Molecular Genetics, Cold Spring Harbor Laboratory, Cold Spring Harbor, N.Y.; Old and Primrose, 1981 Principles of Gene Manipulation, University of California Press, Berkeley; Schleif and Wensink, 1982 Practical Methods in Molecular Biology; Glover (Ed.) 1985 DNA Cloning Vol. I and II, IRL Press, Oxford, UK; Hames and Higgins (Eds.) 1985 Nucleic Acid Hybridization, IRL Press, Oxford, UK; and Setlow and Hollaender 1979 Genetic Engineering: Principles and Methods, Vols. 1-4, Plenum Press, N.Y. Abbreviations and nomenclature, where employed, are deemed standard in the field and commonly used in professional journals such as those cited herein.

The term “transgene” as used herein refers to any polynucleotide that is introduced into the genome of a cell by experimental manipulations. A transgene may be a native DNA or a non-native DNA. “Native” DNA, also referred to as “endogenous” DNA, means a polynucleotide that can naturally exist in the cells of the host species, into which it is introduced. “Non-native” DNA, also referred to as “heterologous” DNA, means a polynucleotide that originates from the cells of a species different from the host species. Non-native DNA may include a native DNA with some modifications that can't be found in the host species.

“Transgenic plant seed” as used herein means a plant seed having a transgene of interest stably incorporated into the seed genome. “Plant seed” may include, but not limited to, inbred seed, F1 hybrid seed produced by crossing a male parental line with a female parental line, F2 seed grown from F1 hybrids, and any seed from a population. “Isoline” or “isogenic line” or “isogenic plant” means the untransformed parental line or any plant seed, from which the transgenic plant of the invention is derived.

The term “plant” as used herein can, depending on context, be understood to refer to whole plants, plant cells, plant organs, plant seeds, and progeny of same. The word “plant” also refers to any plant, including its parts, and may include, but not be limited to, crop plants. Plant parts include, but are not limited to, stems, roots, shoots, fruits, ovules, stamens, leaves, embryos, meristematic regions, callus tissue, gametophytes, sporophytes, pollen, microspores, hypocotyls, cotyledons, anthers, sepals, petals, pollen, seeds, and the like. The class of plants is generally as broad as the class of higher and lower plants amenable to transformation techniques, including angiosperms (monocotyledonous and dicotyledonous plants), gymnosperms, ferns, horsetails, psilophytes, bryophytes, and multicellular algae. The plant can be from a genus selected from the group consisting of Medicago, Lycopersicon, Brassica, Cucumis, Solanum, Juglans, Gossypium, Malus, Vitis, Antirrhinum, Populus, Fragaria, Arabidopsis, Picea, Capsicum, Chenopodium, Dendranthema, Pharbitis, Pinus, Pisum, Oryza, Zea, Triticum, Triticale, Secale, Lolium, Hordeum, Glycine, Pseudotsuga, Kalanchoe, Beta, Helianthus, Nicotiana, Cucurbita, Rosa, Fragaria, Lotus, Medicago, Onobrychis, trifolium, Trigonella, Vigna, Citrus, Linum, Geranium, Manihot, Daucus, Raphanus, Sinapis, Atropa, Datura, Hyoscyamus, Nicotiana, Petunia, Digitalis, Majorana, Ciahorium, Lactuca, Bromus, Asparagus, Antirrhinum, Heterocallis, Nemesis, Pelargonium, Panieum, Pennisetum, Ranunculus, Senecio, Salpiglossis, Browaalia, Phaseolus, Avena, and Allium. “Plants” as used herein can be monocotyledonous crop plants, such as, for example, cereals including wheat (Triticus aestivum), barley (Hordeum vulgare), sorghum (Sorghum bicolor), rye (Secale cereale), triticale, maize (Zea mays), rice (Oryza sativa), sugarcane, and trees including apple, pear, quince, plum, cherry, peach, nectarine, apricot, papaya, mango, poplar, pine, sequoia, cedar, and oak. “Plants” can be dicotyledonous crop plants, such as pea, alfalfa, soybean, carrot, celery, tomato, potato, cotton, tobacco, pepper, oilseed rape, beet, cabbage, cauliflower, broccoli, lettuce and Arabidopsis thaliana.

“Yield” is the harvested grain per land area. For example, in corn, it is generally measured as bushels per acre or tons per hectare.

“Enzymatically active,” when used in reference to the CS protein in accordance with the invention, means that the transgene expressed in the transgenic plant has CS activity.

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

“Amino acid content,” as used herein, means the amount of total amino acids, including free amino acids and bound amino acids in the form of protein. All percentages of amino acids, protein, oil, and starch recited herein are percent dry weight. Amino acids, which are increased in the transgenic plant seed of the invention, are preferably selected from the group consisting of aspartic acid, threonine, glycine, cysteine, valine, methionine, isoleucine, histidine, lysine, arginine, and tryptophan. More preferably, the transgenic plant seed of the invention demonstrates increases over that of the isogenic plant seed of at least 5% in one or more amino acids selected from the group consisting of aspartic acid, threonine, glycine, cysteine, valine, methionine, isoleucine, histidine, lysine, arginine, and tryptophan.

The oil content of the transgenic plant seed of the invention is increased by at least 2% over the oil content of isogenic plant seed. In another embodiment, the oil content of the transgenic plant seed is increased by at least 4% over the oil content of isogenic plant seed. In another embodiment, the oil content of the transgenic plant seed is increased by about 2-10% over the oil content of isogenic plant seed.

The invention encompasses a transgenic plant transformed with an expression vector comprising an isolated polynucleotide. In one embodiment, the polynucleotide of the invention has a sequence as defined in SEQ ID NO:1, 2, 3, 4, 5, 6, 7, 8, 12, 13, 14, or 15. In another embodiment, the polynucleotide encodes a polypeptide having a sequence as defined in SEQ ID NO:16, 17, 18, 19, 22, 23, 24, or 25. In yet another embodiment, a polynucleotide of the invention comprises a polynucleotide which is at least about 50-60%, or at least about 60-70%, or at least about 70-80%, 80-85%, 85-90%, 90-95%, or at least about 95%, 96%, 97%, 98%, 99% or more identical or similar to a polynucleotide having a sequence as defined in SEQ ID NO:1, 2, 3, 4, 5, 6, 7, 8, 12, 13, 14, or 15, or a portion thereof. In yet another embodiment, a polynucleotide of the invention comprises a polynucleotide encoding a polypeptide which is at least about 50-60%, or at least about 60-70%, or at least about 70-80%, 80-85%, 85-90%, 90-95%, or at least about 95%, 96%, 97%, 98%, 99% or more identical or similar to the polypeptide having a sequence as defined in SEQ ID NO:16, 17, 18, 19, 22, 23, 24, or 25. The sequence identity and sequence similarity are defined as below.

One of the embodiments encompasses allelic variants of a polynucleotide having a sequence as defined in SEQ ID NO:1, 2, 3, 4, 5, 6, 7, 8, 12, 13, 14, or 15, or a polynucleotide encoding a polypeptide having a sequence as defined in SEQ ID NO:16, 17, 18, 19, 22, 23, 24, or 25. As used herein, the term “allelic variant” refers to a polynucleotide containing polymorphisms that lead to changes in the amino acid sequences of a protein encoded by the nucleotide and that exist within a natural population (e.g., a plant species or variety). Such natural allelic variations can typically result in 1-5% variance in a polynucleotide encoding a protein, or 1-5% variance in the encoded protein. Allelic variants can be identified by sequencing the nucleic acid of interest in a number of different plants, which can be readily carried out by using, for example, hybridization probes to identify the same gene genetic locus in those plants. Any and all such nucleic acid variations in a polynucleotide and resulting amino acid polymorphisms or variations of a protein that are the result of natural allelic variation and that do not alter the functional activity of the encoded protein, are intended to be within the scope of the invention.

As used herein, the term “hybridizes under stringent conditions” is intended to describe conditions for hybridization and washing under which nucleotide sequences at least 60% similar or identical to each other typically remain hybridized to each other. In another embodiment, the conditions are such that sequences at least about 65%, or at least about 70%, or at least about 75%, or at least about 80%, or more similar or identical to each other typically remain hybridized to each other. Such stringent conditions are known to those skilled in the art and described as below. A preferred, non-limiting example of stringent conditions are hybridization in 6X sodium chloride/sodium citrate (SSC) at about 45° C., followed by one or more washes in 0.2×SSC, 0.1% SDS at 50-65° C.

In yet another embodiment, an isolated nucleic acid is complementary to a polynucleotide as defined in SEQ ID NO:1, 2, 3, 4, 5, 6, 7, 8, 12, 13, 14, or 15, or a polynucleotide encoding a polypeptide having a sequence as defined in SEQ ID NO:16, 17, 18, 19, 22, 23, 24, or 25, or a polynucleotide having 70% sequence identity to a polynucleotide as defined in SEQ ID NO:1, 2, 3, 4, 5, 6, 7, 8, 12, 13, 14, or 15, or a polynucleotide encoding a polypeptide having 70% sequence identity to a polypeptide as defined in SEQ ID NO:16, 17, 18, 19, 22, 23, 24, or 25, or a polynucleotide hybridizing to a polynucleotide as defined in SEQ ID NO:1, 2, 3, 4, 5, 6, 7, 8, 12, 13, 14, or 15, or a polynucleotide hybridizing to a polynucleotide encoding a polypeptide having a sequence as defined in SEQ ID NO:16, 17, 18, 19, 22, 23, 24, or 25. As used herein, “complementary” polynucleotides refer to those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA.

In another embodiment, the polynucleotides of the invention comprise a polynucleotide having a sequence as defined in SEQ ID NO:1, 2, 3, 4, 5, 6, 7, 8, 12, 13, 14, or 15, or a polynucleotide encoding a polypeptide having a sequence as defined in SEQ ID NO:16, 17, 18, 19, 22, 23, 24, or 25, or any of the polynucleotide homologs aforementioned, wherein the polynucleotides encode CS that confer an economically relevant trait in a plant. Moreover, the polynucleotides of the invention can comprise only a portion of the coding region of a polynucleotide sequence as defined in SEQ ID NO:1, 2, 3, 4, 5, 6, 7, 8, 12, 13, 14, or 15, or a polynucleotide encoding a polypeptide having a sequence as defined in SEQ ID NO:16, 17, 18, 19, 22, 23, 24, or 25, or the homologs thereof, for example, a fragment which can be used as a probe or primer

The transgenic plant seed of the invention may be produced by transforming the CS gene into a plant using any known method of transforming a monocot or dicot. A variety of methods for introducing polynucleotides into the genome of plants and for the regeneration of plants from plant tissues or plant cells are known. See e.g., Plant Molecular Biology and Biotechnology (CRC Press, Boca Raton, Fla.), chapter 6/7, pp. 71-119 (1993); White F F (1993) Vectors for Gene Transfer in Higher Plants; Transgenic Plants, vol. 1, Engineering and Utilization, Ed.: Kung and Wu R, Academic Press, 15-38; Jenes B et al. (1993) Techniques for Gene Transfer; Transgenic Plants, vol. 1, Engineering and Utilization, Ed.: Kung and R. Wu, Academic Press, pp. 128-143; Potrykus (1991) Annu Rev Plant Physiol Plant Molec Biol 42:205-225; Halford N G, Shewry P R (2000) Br Med Bull 56(1):62-73.

Transformation methods may include direct and indirect methods of transformation. Suitable direct methods include polyethylene glycol induced DNA uptake, liposome-mediated transformation (US 4,536,475), biolistic methods using the gene gun (Fromm M E et al., Bio/Technology. 8(9):833-9, 1990; Gordon-Kamm et al., Plant Cell 2:603, 1990), electroporation, incubation of dry embryos in DNA-comprising solution, and microinjection. In the case of these direct transformation methods, the plasmid used need not meet any particular requirements. Simple plasmids, such as those of the pUC series, pBR322, M13mp series, and the like can be used. If intact plants are to be regenerated from the transformed cells, an additional selectable marker gene is preferably located on the plasmid. The direct transformation techniques are equally suitable for dicotyledonous and monocotyledonous plants.

Transformation can also be carried out by bacterial infection by means of Agrobacterium (EP 0 116 718), viral infection by means of viral vectors (EP 0 067 553; US 4,407,956; WO 95/34668; WO 93/03161) or by means of pollen (EP 0 270 356; WO 85/01856; US 4,684,611). Agrobacterium based transformation techniques are well known in the art. The Agrobacterium strain (e.g., Agrobacterium tumefaciens or Agrobacterium rhizogenes) comprises a plasmid (Ti or Ri plasmid) and a T-DNA element which is transferred to the plant following infection with Agrobacterium. The

T-DNA (transferred DNA) is integrated into the genome of the plant cell. The T-DNA may be localized on the Ri- or Ti-plasmid or is separately comprised in a so-called binary vector. Methods for the Agrobacterium-mediated transformation are described, for example, in Horsch R B et al. (1985) Science 225:1229. The transformation of plants by Agrobacteria is described in, for example, White F F, Vectors for Gene Transfer in Higher Plants, Transgenic Plants, Vol. 1, Engineering and Utilization, edited by S. D. Kung and R. Wu, Academic Press, 1993, pp. 15-38; Jenes B et al. Techniques for Gene Transfer, Transgenic Plants, Vol. 1, Engineering and Utilization, edited by S.D. Kung and R. Wu, Academic Press, 1993, pp. 128-143; Potrykus (1991) Annu Rev Plant Physiol Plant Molec Biol 42:205-225.

The CS gene may be transformed into a corn plant using particle bombardment as set forth in U.S. Pat. Nos. 4,945,050; 5,036,006; 5,100,792; 5,302,523; 5,464,765; 5,120,657; 6,084,154; and the like. The transgenic corn seed of the invention may be made using Agrobacterium transformation, as described in U.S. Pat. Nos. 5,591,616; 5,731,179; 5,981,840; 6,162,965; 6,420,630, U.S. patent application publication number 2002/0104132, and the like. Alternatively, the transgenic corn seed of the invention may be produced using plastid transformation methods suitable for use in corn. Plastid transformation in tobacco is described, for example, in Zoubenko, et al. (1994) Nucleic Acids Res. 22, 3819-3824; Ruf, et al. (2001) Nature Biotechnol. 19, 870-875; Kuroda et al. (2001) Plant Physiol. 125, 430-436; Kuroda et al. (2001) Nucleic Acids Res. 29, 970-975; Hajdukiewica et al. (2001) Plant J. 27, 161-170; and Corneille, et al. (2001) Plant J. 72, 171-178. Additional plastid transformation methods employing the phiC31 phage integrase are disclosed in Lutz, et al. (2004) The Plant J. 37, 906. Additional transformation methods include, but are not limited to, the following starting materials and methods in Table 1:

TABLE 1

| Variety | Material/Citation |
|---|---|
| Monocotyledonous | Immature embryos (EP-A1 672 752) |
| plants: | Callus (EP-A1 604 662) |
| | Embryogenic callus (U.S. Pat. No. 6,074,877) |
| | Inflorescence (U.S. Pat. No. 6,037,522) |
| | Flower (in planta) (WO 01/12828) |
| Banana | U.S. Pat. No. 5,792,935; EP-A1 731 632; |
| | U.S. Pat. No. 6,133,035 |
| Barley | WO 99/04618 |
| Maize | U.S. Pat. No. 5,177,010; U.S. Pat. No. 5,987,840 |
| Pineapple | U.S. Pat. No. 5,952,543; WO 01/33943 |
| Rice | EP-A1 897 013; U.S. Pat. No. 6,215,051; |
| | WO 01/12828 |
| Wheat | AU-B 738 153; EP-A1 856 060 |
| Beans | U.S. Pat. No. 5,169,770; EP-A1 397 687 |
| Brassica | U.S. Pat. No. 5,188,958; EP-A1 270 615; |
| | EP-A1 1,009,845 |
| Cacao | U.S. Pat. No. 6,150,587 |
| Citrus | U.S. Pat. No. 6,103,955 |
| Coffee | AU 729 635 |
| Cotton | U.S. Pat. No. 5,004,863; EP-A1 270 355; |
| | U.S. Pat. No. 5,846,797; EP-A1 1,183,377; |
| | EP-A1 1,050,334; EP-A1 1,197,579; |
| | EP-A1 1,159,436 |
| | Pollen transformation (U.S. Pat. No. 5,929,300) |
| | In planta transformation (U.S. Pat. No. 5,994,624) |
| Pea | U.S. Pat. No. 5,286,635 |
| Pepper | U.S. Pat. No. 5,262,316 |
| Poplar | U.S. Pat. No. 4,795,855 |
| Soybean | cotyledonary node of germinated soybean seedlings |
| | shoot apex (U.S. Pat. No. 5,164,310) |
| | axillary meristematic tissue of primary, or higher |
| | leaf node of about 7 days germinated |
| | soybean seedlings organogenic callus |
| | cultures dehydrated embryo axes |
| | U.S. Pat. No. 5,376,543; EP-A1 397 687; |
| | U.S. Pat. No. 5,416,011; U.S. Pat. No. 5,968,830; |
| | U.S. Pat. No. 5,563,055; U.S. Pat. No. 5,959,179; |
| | EP-A1 652 965; EP-A1 1,141,346 |
| Sugarbeet | EP-A1 517 833; WO 01/42480 |
| Tomato | U.S. Pat. No. 5,565,347 |

In accordance with the invention, the polynucleotide encoding the CS gene may be present in any expression cassette suitable for expression of a gene in a plant. Such an expression cassette comprises one or more transcription regulatory elements operably linked to one or more polynucleotides of the invention. The expression cassette may comprise a polynucleotide encoding a cell compartment transit peptide, such as a plastid transit peptide. In one embodiment, the transcription regulatory element is a promoter capable of regulating constitutive expression of an operably linked polynucleotide. A “constitutive promoter” refers to a promoter that is able to express the open reading frame or the regulatory element that it controls in all or nearly all of the plant tissues during all or nearly all developmental stages of the plant. Constitutive promoters include, but not limited to, the 35S CaMV promoter from plant viruses (Franck et al., Cell 21:285-294, 1980), the Nos promoter (An G. at al., The Plant Cell 3:225-233, 1990), the ubiquitin promoter (Christensen et al., Plant Mol. Biol. 12:619-632, 1992 and 18:581-8, 1991), the MAS promoter (Velten et al., EMBO J. 3:2723-30, 1984), the maize H3 histone promoter (Lepetit et al., Mol Gen. Genet 231:276-85, 1992), the ALS promoter (WO96/30530), the 19S CaMV promoter (U.S. Pat. No. 5,352,605), the super-promoter (U.S. Pat. No. 5,955,646), the figwort mosaic virus promoter (U.S. Pat. No. 6,051,753), the rice actin promoter (U.S. Pat. No. 5,641,876), and the Rubisco small subunit promoter (U.S. Pat. No. 4,962,028).

A “tissue-specific promoter” or “tissue-preferred promoter” refers to a regulated promoter that is not expressed in all plant cells but only in one or more cell types in specific organs (such as leaves or seeds), specific tissues (such as embryo, endosperm, or cotyledon), or specific cell types (such as leaf parenchyma or seed storage cells). There also include promoters that are temporally regulated, such as in early or late embryogenesis, during fruit ripening in developing seeds or fruit, in fully differentiated leaf, or at the onset of senescence. Suitable promoters include the napin-gene promoter from rapeseed (U.S. Pat. No. 5,608,152), the USP-promoter from Vicia faba (Baeumlein et al., Mol Gen Genet. 225(3):459-67, 1991), the oleosin-promoter from Arabidopsis (WO 98/45461), the phaseolin-promoter from Phaseolus vulgaris (U.S. Pat. No. 5,504,200), the Bce4-promoter from Brassica (WO 91/13980) or the legumin B4 promoter (LeB4; Baeumlein et al., Plant Journal, 2(2):233-9, 1992) as well as promoters conferring seed specific expression in monocot plants like maize, barley, wheat, rye, rice, such as a maize branching enzyme 2b promoter (Kim et al., Plant Mol. Boil.38:945-956, 1998), or a maize shrunken-2 promoter (Russel and Fromm, Transgenic Research 6(2):157-168, 1997), or a maize granule bound starch synthase promoter (Russel and Fromm, Transgenic Research 6(2):157-168, 1997), or promoters of maize starch synthase I (Knight et al, Plant J 14 (5):613-622, 1998) and rice starch synthase I (Tanaka et al, Plant Physiol. 108 (2):677-683, 1995). Other suitable promoters to note are the Ipt2 or Ipt1-gene promoter from barley (WO 95/15389 and WO 95/23230) or those described in WO 99/16890 (promoters from the barley hordein-gene, rice glutelin gene, rice oryzin gene, rice prolamin gene, wheat gliadin gene, wheat glutelin gene, maize zein gene, oat glutelin gene, Sorghum kasirin-gene and rye secalin gene). Endosperm-specific promoters include, for example, a maize 10 kD zein promoter (Kirihara et al., Gene, 71:359-370), or a maize 27 kD zein promoter (Russel and Fromm, Transgenic Research 6(2):157-168, 1997). Promoters suitable for preferential expression in plant root tissues include, for example, the promoter derived from corn nicotianamine synthase gene (US 2003/0131377) and rice RCC3 promoter (US 2006/0101541). Suitable promoter for preferential expression in plant green tissues include the promoters from genes such as maize aldolase gene FDA (US 2004/0216189), aldolase and pyruvate orthophosphate dikinase (PPDK) (Taniguchi et. al., Plant Cell Physiol. 41(1):42-48, 2000).

Nucleotide sequences encoding plastid transit peptides are well known in the art, as disclosed, for example, in U.S. Pat. Nos. 5,717,084; 5,728,925; 6,063,601; 6,130,366; and the like. Cell compartment transit peptides include, but are not limited to, the ferredoxin transit peptide and the starch branching enzyme 2b transit peptide. The expression cassette that includes the CS gene may also contain suitable termination sequences and other regulatory sequences, which may optimize expression of the gene in the plant.

The term “sequence identity” or “identity” in the context of two nucleic acid or polypeptide sequences makes reference to those positions in the two sequences where identical pairs of symbols fall together when the sequences are aligned for maximum correspondence over a specified comparison window, for example, either the entire sequence as in a global alignment or less than the entire sequence as in a local alignment. In protein sequence alignment, amino acid residues at the same position are considered conserved when the amino acid residues have similar chemical properties (e.g., charge or hydrophobicity). The sequences that differ by such conservative substitutions are said to have “sequence similarity” or “similarity”. Sequence similarity may be altered without affecting protein function. Means for making this adjustment are well known to those of skilled in the art. Typically this involves scoring a conservative substitution as a partial match rather than a mismatch, thereby increasing the percentage of sequence similarity.

As used herein, “percentage of sequence identity” or “sequence identity percentage” denotes a value determined by first noting in two optimally aligned sequences over a comparison window, either globally or locally, at each constituent position as to whether the identical nucleic acid base or amino acid residue occurs in both sequences, denoted as a match, or does not occur in both sequences, denoted as a mismatch. As said alignments are constructed by optimizing the number of matching bases, while concurrently allowing both for mismatches at any position and for the introduction of arbitrarily-sized gaps, or null or empty regions where to do so increases the significance or quality of the alignment, the calculation determines the total number of positions for which the match condition exists, and then divides this number by the total number of positions in the window of comparison, and lastly multiplies the result by 100 to yield the percentage of sequence identity. “Percentage of sequence similarity” for protein sequences can be calculated using the same principle, wherein the conservative substitution is calculated as a partial rather than a complete mismatch. Thus, for example, where an identical amino acid is given a score of 1 and a non-conservative substitution is given a score of zero, a conservative substitution is given a score between zero and 1. The scoring of conservative substitutions can be obtained from amino acid matrices known in the art, for example, Blosum or PAM matrices.

Methods of alignment of sequences for comparison are well known in the art. The determination of percent identity or percent similarity (for proteins) between two sequences can be accomplished using a mathematical algorithm. Preferred, non-limiting examples of such mathematical algorithms are, the algorithm of Myers and Miller (Bioinformatics, 4(1):11-17, 1988), the Needleman-Wunsch global alignment (J Mol Biol. 48(3):443-53, 1970), the Smith-Waterman local alignment (J. Mol. Biol., 147:195-197, 1981), the search-for-similarity-method of Pearson and Lipman (PNAS, 85(8): 2444-2448, 1988), the algorithm of Karlin and Altschul (J. Mol. Biol., 215(3):403-410, 1990; PNAS, 90:5873-5877,1993). Computer implementations of these mathematical algorithms can be utilized for comparison of sequences to determine sequence identity or to identify homologs. Such implementations include, but are not limited to, the programs described below.

The term “sequence alignment” used herein refers to the result of applying one of several methods of arranging the primary sequences of DNA, RNA, or protein to identify regions of similarity that may be a consequence of functional, structural, or evolutionary relationships between the sequences. Computational approaches to sequence alignment generally fall into two categories: global alignments and local alignments. A global alignment is constrained to fully contain each constituent sequence, while a local alignment is free to identify any sub-regions of similarity between the given sequences, and which otherwise can be quite dissimilar. Multiple alignments (e.g., of more than two DNA or protein sequences) can be performed using the ClustalW algorithm (Thompson et. al. ClustalW: improving the sensitivity of progressive multiple sequence alignment through sequence weighting, position-specific gap penalties and weight matrix choice. Nucleic Acids Res. 22:4673-4680, 1994) as implemented in, for example, Vector NTI package (Invitrogen, 1600 Faraday Ave., Carlsbad, Calif. 92008).

It is well known in the art that one or more amino acids in a native sequence can be substituted with another amino acid(s), the charge and polarity of which are similar to that of the native amino acid, i.e., a conservative amino acid substitution. Conserved substitutions for an amino acid within the native polypeptide sequence can be selected from other members of the class to which the naturally occurring amino acid belongs. Amino acids can be divided into the following four groups: (1) acidic amino acids, (2) basic amino acids, (3) neutral polar amino acids, and (4) neutral nonpolar amino acids. Representative amino acids within these various groups include, but are not limited to: (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, tyrosine, asparagine, and glutamine; and (4) neutral nonpolar (hydrophobic) amino acids such as alanine, leucine, isoleucine, valine, proline, phenylalanine, tryptophan, and methionine.

A typical codon usage of an organism tends to be different from that of another. Different codon usage is well known to affect the expression of a non-native gene when introduced into a foreign genome that has a different codon usage. The information usually used for the optimization process is the DNA or protein sequence to be optimized and a codon usage table (which is often referred to as the reference set) of the host organism. Codon optimization basically involves altering the rare codons in the target gene so that they more closely reflect the codon usage of the host organism without modifying the amino acid sequence of the encoded protein (Gustafsson et al., Trends Biotechnol. 22: 346-353, 2004).

The potential for reducing costs associated with meat production using the transgenic corn seed of the invention is great. The improved amino acid profile of the transgenic corn of the invention allows it to be used in feed without soybean meal supplementation, thus eliminating the expense and environmental impact associated with feeds containing soybean meal. Moreover, the improved oil content of the transgenic corn seed of the invention will allow animal feed producers to minimize use of animal by-products as additives to animal feed, thus minimizing possible contamination of the human food chain with infectious agents such as the bovine spongiform encephalopathy agent. Farmers will be able to obtain a more optimal feed conversion ratio using the transgenic corn of the invention than is possible through feeding yellow dent corn. The transgenic corn seed of the invention is therefore particularly useful as animal feed.

Identity preservation is a method to segregate a specific product during production and storage and transportation to deliver the product the customer needs. This is a way to capture the added value of a unique product.

Traceability is ability to trace the history, application or location of materials under consideration. The material can be a transgenic seed, a chemical ingredient or a transgenic DNA or transgenic protein. For example, it can be the specific CS protein or DNA to be traced. This can be useful to ensure food safety and/or value capturing.

The invention is further illustrated by the following examples, which are not to be construed in any way as imposing limitations upon the scope thereof.

### EXAMPLES

### Example 1

### CS Gene Synthesis and Codon Optimization for Corn Expression

CS DNA sequences from E. coli and S. cerevisiae were optimized for expression in corn and de novo synthesized by methods known to those of skill in the art (Gustafsson et al., Trends Biotechnol. 22: 346-353, 2004). Codons encoding amino acid sequence of each CS were optimized by iteratively sampling from corn codon usage table to find a low free energy solution, resulting in decreased secondary structure of the mRNA. The codon optimized gene sequences are SEQ ID NOs:2, 4, 6, 8, 10, and 11.

### Example 2

### Construction of Transgenic Expression Cassette and Super-Binary Vector

The plasmid vector SB11 (Komari et al., Plant Journal 10(1): 165-74, 1996) was used as a base vector to generate the plasmid vector pEXS1000. The ZmAHASL2 promoter::ZmAHASL2 gene::ZmAHASL2 3′UTR terminator cassette was inserted between the left border repeat and the right border repeat of the plasmid vector SB11. Acetohydroxyacid synthase, or “AHAS”, and sequences and constructs comprising the AHAS sequences are described in U.S. Pat. No. 6,653,529. The gene cassettes containing promoter::trait gene of interest::NOS terminator were inserted into plasmid vector pEXS1000 in order to generate the plasmid vectors for recombination with plasmid vector SB11 prior to plant transformation. The constructs as shown in Table 2 were made for corn transformation. These constructs were transformed to a maize inbred line by agrobacterium-mediated transformation, using AHAS as a selection marker (Fang et al., Plant Molecular Biology 18(6): 1185-1187, 1992).

TABLE 2
List of CS constructs for plant transformation

| Construct | Gene components |
|---|---|
| CS1001 | Maize 10 kD zein promoter::Ferredoxin transit peptide::corn-codon |
| | optimized Yeast CS1 (SEQ ID NO. 4)::Nos terminator |
| CS1002 | Maize 10 kD zein promoter::Ferredoxin transit peptide:: corn-codon |
| | optimized E. coli CS1 (SEQ ID NO. 2)::Nos terminator |
| CS1003 | Maize shrunken-2 promoter::Maize starch branching enzyme 2b transit |
| | peptide:: corn-codon optimized Yeast CS1 (SEQ ID NO. 4)::Nos terminator |
| CS1004 | Maize shrunken-2 promoter:: Maize starch branching enzyme 2b transit |
| | peptide:: corn-codon optimized E. coli CS1 (SEQ ID NO. 2)::Nos terminator |
| CS1005 | Maize 10 kD zein promoter::Ferredoxin transit peptide:: corn-codon |
| | optimized Anabaena CS (SEQ ID NO. 8)::Nos terminator |
| CS1006 | Maize 10 kD zein promoter::Mitochondrial signal peptide:: corn-codon |
| | optimized Yeast CS1 (SEQ ID NO. 4)::Nos terminator |
| CS1007 | Maize shrunken-2 promoter::Ferredoxin transit peptide::corn-codon |
| | optimized Anabaena CS (SEQ ID NO. 8)::Nos terminator |
| CS1008 | Maize 10 kD zein promoter::Ferredoxin transit peptide::corn-codon |
| | optimized Yeast CS2 (SEQ ID NO. 6)::Nos terminator |
| CS1009 | Maize shrunken-2 promoter::Ferredoxin transit peptide:: corn-codon |
| | optimized Yeast CS2 (SEQ ID NO. 6)::Nos terminator |
| CS1010 | Maize starch synthase I promoter:: Maize starch branching enzyme 2b |
| | transit peptide:: corn-codon optimized Yeast CS1 (SEQ ID NO. 4)::Nos |
| | terminator |
| CS1011 | Maize shrunken-2 promoter:: corn-codon optimized Yeast CS1 (SEQ ID |
| | NO. 4)::Nos terminator |
| CS1012 | Maize granule bound starch synthase promoter::Ferredoxin transit peptide:: |
| | corn-codon optimized Yeast CS1 (SEQ ID NO. 4)::Nos terminator |
| CS1013 | Maize shrunken-2 promoter::Pumpkin glyoxysomal signal peptide:: corn- |
| | codon optimized Pumpkin glyoxysomal CS (SEQ ID NO. 10)::Nos |
| | terminator |
| CS1014 | Rice starch synthase I promoter::Ferredoxin transit peptide:: corn-codon |
| | optimized Yeast CS1 (SEQ ID NO. 4)::Nos terminator |
| CS1015 | Maize 10 kD zein promoter::Ferredoxin transit peptide::corn-codon |
| | optimized Pumpkin glyoxysomal CS (SEQ ID NO. 10)::Nos terminator |

### Example 3

### Maize transformation

Agrobacterium cells harboring a plasmid containing the gene of interest and the maize mutated AHAS gene were grown in YP medium supplemented with appropriate antibiotics for 1-2 days. One loop of Agrobacterium cells were collected and suspended in 1.8 ml M-LS-002 medium (LS-inf). The cultures were incubated with shaking at 1,200 rpm for 5 min-3 hrs. Corn cobs were harvested at 8-11 days after pollination. The cobs were sterilized in 20% Clorox solution for 5 min, followed by spraying with 70% Ethanol and then thoroughly rinsing with sterile water. Immature embryos 0.8-2.0 mm in size were dissected into the tube containing Agrobacterium cells in LS-inf solution.

Agrobacterium infection of the embryos was carried out by inverting the tube several times. The mixture was poured onto a filter paper disk on the surface of a plate containing co-cultivation medium (M-LS-011). The liquid agro-solution was removed and the embryos were checked under a microscope and placed scutellum side up. Embryos were cultured in the dark at 22° C. for 2-4 days, and were transferred to M-MS-101 medium without selection and incubated for four to seven days. Embryos were then transferred to M-LS-202 medium containing 0.75μM imazethapyr and grown for four weeks at 27° C. to select for transformed callus cells.

Plant regeneration was initiated by transferring resistant calli to M-LS-504 medium supplemented with 0.75 μM imazethapyr and grown under light at 26° C. for two to three weeks. Regenerated shoots were then transferred to a rooting box with M-MS-618 medium (0.5 μM imazethapyr). Plantlets with roots were transferred to soil-less potting mixture and grown in a growth chamber for a week, then transplanted to larger pots and maintained in a greenhouse until maturity.

### Example 4

### Analysis of CS Expression in Transgenic Plants—Citrate Synthase Assay

Utilizing T3, T4, or T5 ears, five kernels from a frozen ear harvested at 23 days after pollination (DAP) were first ground to a dry powder in a −20° C. chilled mortar and then into a slurry after addition of 5m1 of ice-cold Tris extraction buffer (50 mM Tris-HCl pH 8.0, 5 mM EDTA, 10% glycerol). Insoluble debris was removed by centrifugation at 13,000 g and 4° C. for 5 min. The supernatant was used for enzyme assay. Citrate synthase activity was assayed by measuring production of CoA through reaction with dithiobis-(2-nitrobenzoate) (DTNB) as described by Srere, P. (Meth Enzymol 3:3-11, 1969). An enzyme assay master mix was prepared using 19 μof supernatant in a total volume of 1862 μl of 50 mM Tris-HCl pH 8.0, 0.25 mM DTNB and 0.25 mM acetyl-CoA. Quadruplicate reactions were started in aliquots (200 μl) of master mix with 0.5 mM oxaloacetic acid (OAA) or with water in quadruplicate control reactions. The assays were proceeded at 30° C. for 4 minutes and were terminated at 95° C. The volume was adjusted to 600 μl and the absorbance was measured at 412 nm. Activities were calculated based on the absorbance difference of assays performed in the presence or absence of the substrate OAA. Protein concentrations were determined by Bradford's dye-binding assay.

Because there is native maize CS activity, the transgenic CS was separated from native maize CS by FPLC to confirm the expression of transgenic CS protein, using anion exchange chromatography. Maize kernels at 23 DAP were ground in an ice-cooled mortar (40 kernels in 20 ml) with extraction buffer (50 mM Tris-HCl pH 8.0, 5 mM EDTA, 2% PEG-8000). The suspension was clarified at 9,500×g and 4° C. for 30 min and the supernatant adjusted to 20% PEG-8000. The proteins that precipitated after 60 min on ice were recovered at 25,000×g and 4° C. for 20 min. and resuspended in 10 ml of buffer A (50 mM Tris-HCl pH 8.0). Resuspended samples were clarified at 25,000g and 4° C. for 10 min and loaded onto a MonoQ™ HR10/10 column (GE Healthcare) at 1-2 ml/min. Proteins were eluted with a 50 ml linear gradient up to 50% buffer B (50 mM Tris-HCl pH 8.0, 1 M NaCl) and 1 ml fractions were collected. Citrate synthase activity (CoA production) was monitored in column fractions through reaction with dithiobis-(2-nitrobenzoate) (DTNB) essentially as described (Srere, P. 1969. Meth Enzymol 3:3-11).

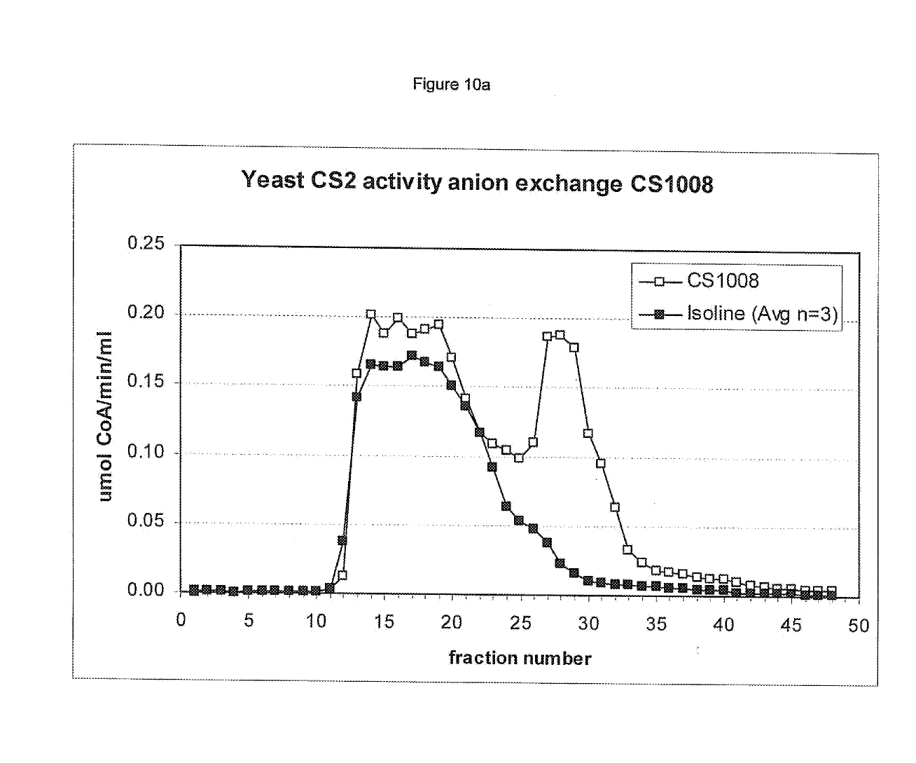

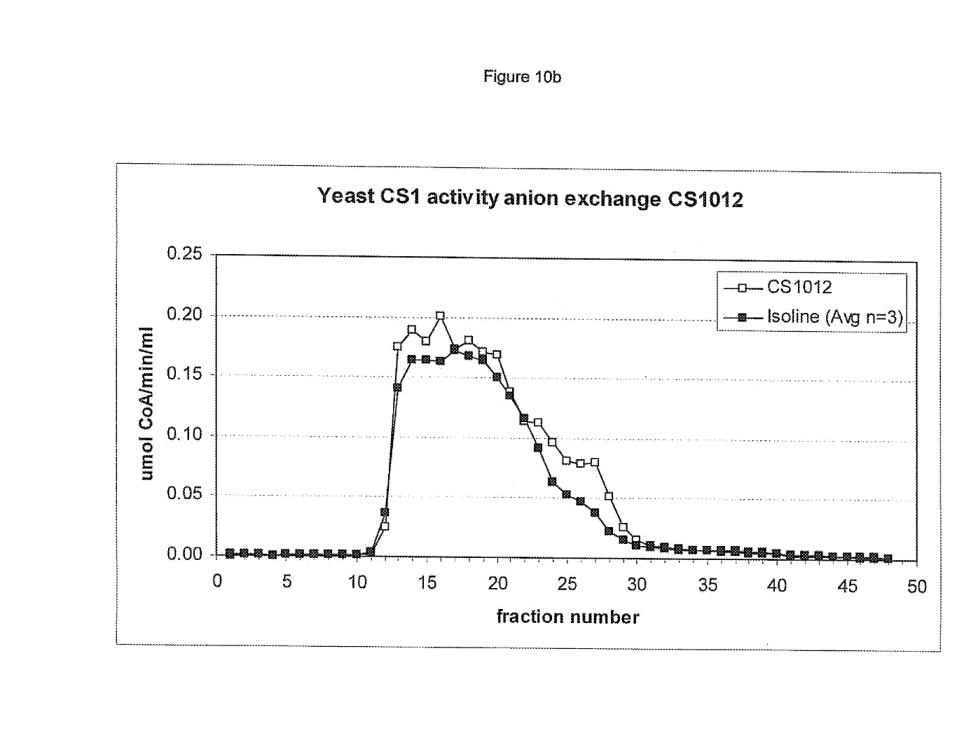

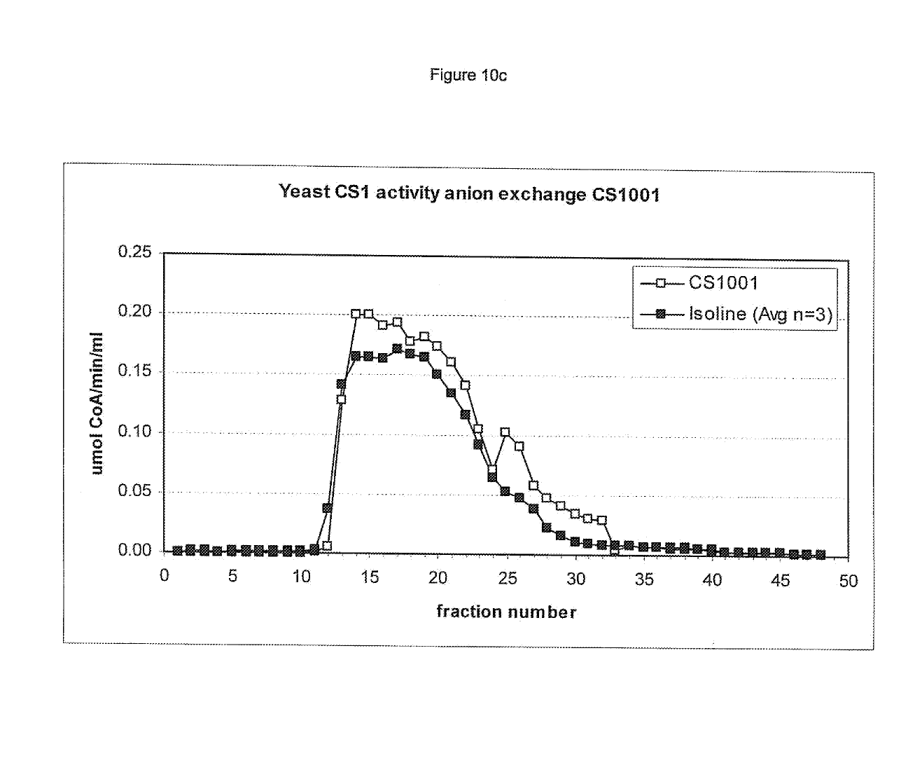

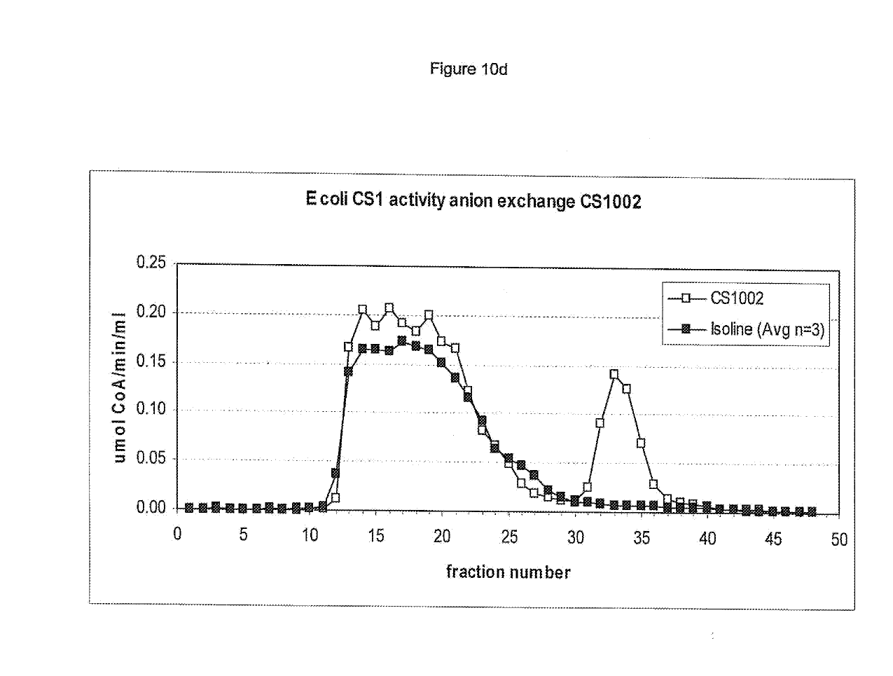

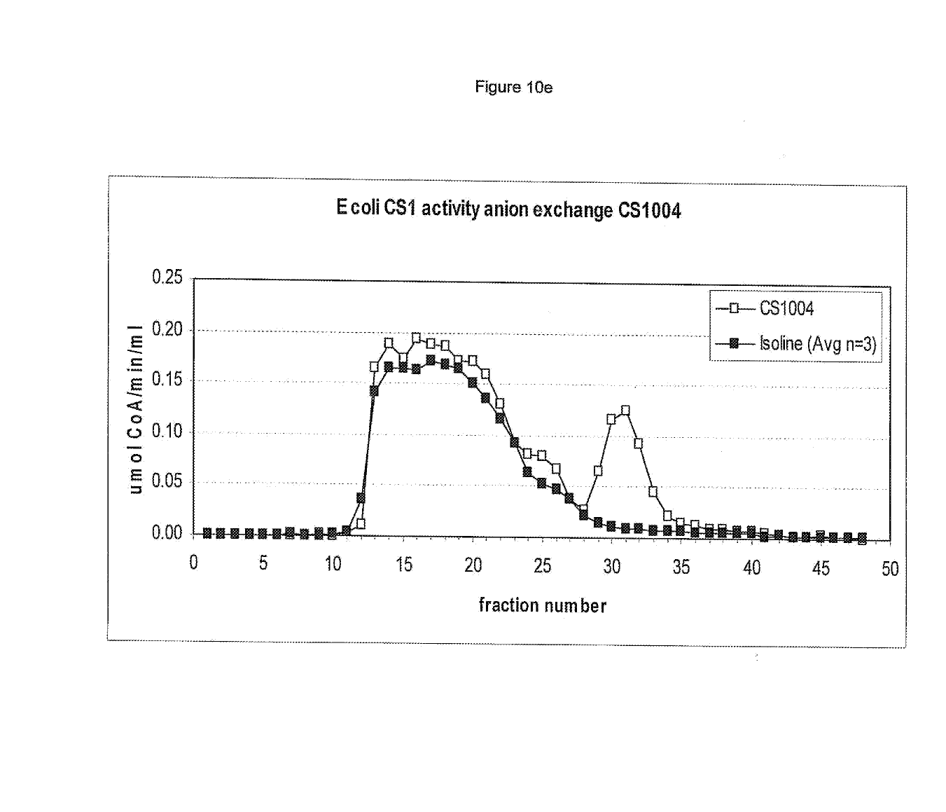

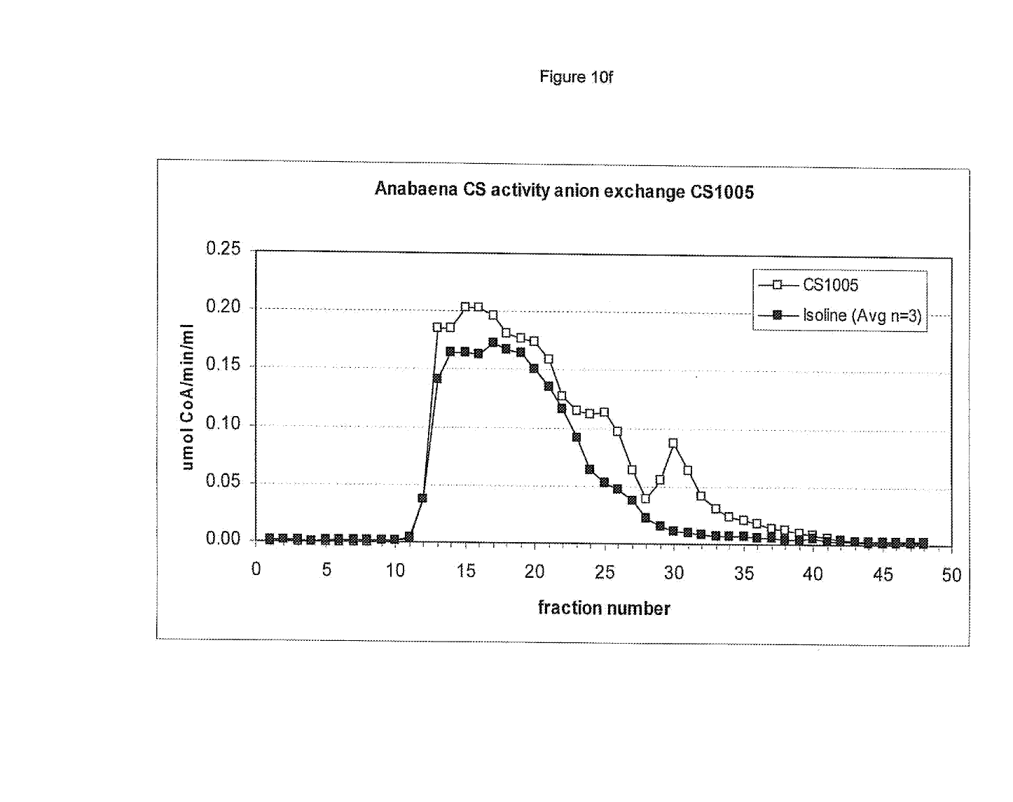

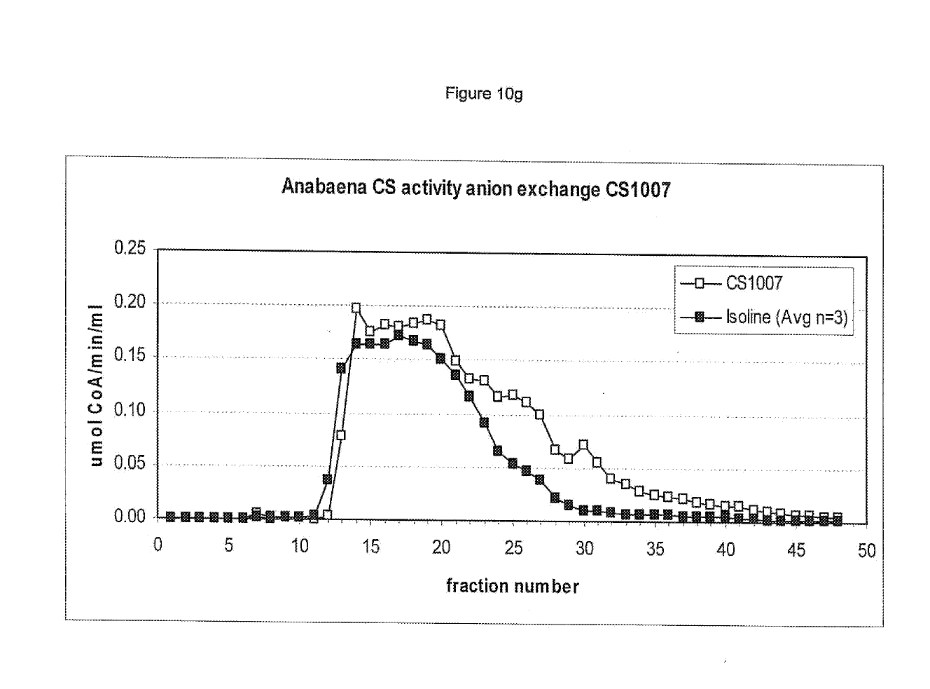

FIGS. 10a-g contain graphs showing the fraction numbers with CS activity (μmol CoA/min/ml) in each fraction for constructs CS1008, CS1012, CS1001, CS1002, CS1004, CS1005, and CS1007, respectively. Each transgenic CS showed an activity peak in addition to the maize CS activity peak (FIG. 45a-g).

### Example 5

### Amino Acid, Protein and Oil Analysis of Transgenic Seeds

Transgenic T1 seeds containing a CS gene were planted in a summer nursery. The T2 plants were screened for transgene zygosity by quantitative PCR of leaf DNA. Homozygous plants were self-pollinated. Mature T2 seeds from homozygous plants were pooled and used for grain composition analysis. Mature seed samples were ground with an IKA® A11 basic analytical mill (IKA® Works, Inc., Wilmington, N.C.). The samples were re-ground and analyzed for complete amino acid profile (AAP) using the method described in Association of Official Analytical Chemists (AOAC) Official Method 982.30 E (a, b, c), CHP 45.3.05, 2000. The samples were also analyzed for crude protein (Combustion Analysis (LECO) AOAC Official Method 990.03, 2000), crude fat (Ether Extraction, AOAC Official Method 920.39 (A), 2000), and moisture (vacuum oven, AOAC Official Method 934.01, 2000).

The grain composition analysis, shown in FIG. 11, demonstrates that plants expressing a heterologous CS protein had enhanced grain nutrient contents in T2 lines. The results shown in FIG. 11 have clearly demonstrated the following:

1. Plants containing a heterologous CS gene from different organisms such as yeast CS1 and CS2, E. coli CS1, or pumpkin glyoxysome CS, targeted to the plastid, mitochondria, cytosol, or glyoxysome of corn seed, showed at least 5% increases in protein and/or multiple essential amino acid contents in the grain, such as cysteine and valine. For example, in comparison to grain of wild-type isoline, the data generated from 8 events expressing yeast CS1 gene in the plastid showed a 11.4 and 12.9% increase respectively for cysteine and valine (FIG. 11).

2. Targeting the expression of a heterologous CS gene in different cell compartments can have an impact on grain nutrition enhancement. FIG. 11 shows that for increasing grain nutrition, a heterologous CS is preferably expressed in an intracellular compartment such as the cytosol, the mitochondria, or the plastid; most preferably, a heterologous CS is expressed in the plastids.

3. FIG. 11 indicates that the promoter used to drive the expression of a heterologous CS in corn seed can have an impact on grain nutrition enhancement. For example, using either maize 10 kD zein promoter or maize Shrunken-2 (Sh-2) promoter to drive the expression of yeast CS1 in the plastid showed a greater increase in grain nutrition than using maize granule bound starch synthase (GBSS) promoter.

### Example 6

### Field Test of Transgenic Hybrid

A transgenic corn inbred containing homozygous transgene (CS) was crossed with a proprietary inbred to make F1 hybrid. The transgenic hybrids along with the wild type control hybrid were planted in six locations with 3 replicates per location for yield test. For grain composition analysis, the transgenic hybrids were planted in 3 locations with 6 replicates per location. Six plants per hybrid were hand-pollinated. Three well-pollinated ears were selected and pooled for grain composition analysis. Oil and protein contents were assayed by NIR methods known to those of skill in the art. See for example, Givens et al (1997) Nutrition Research Reviews 10: 83-114.

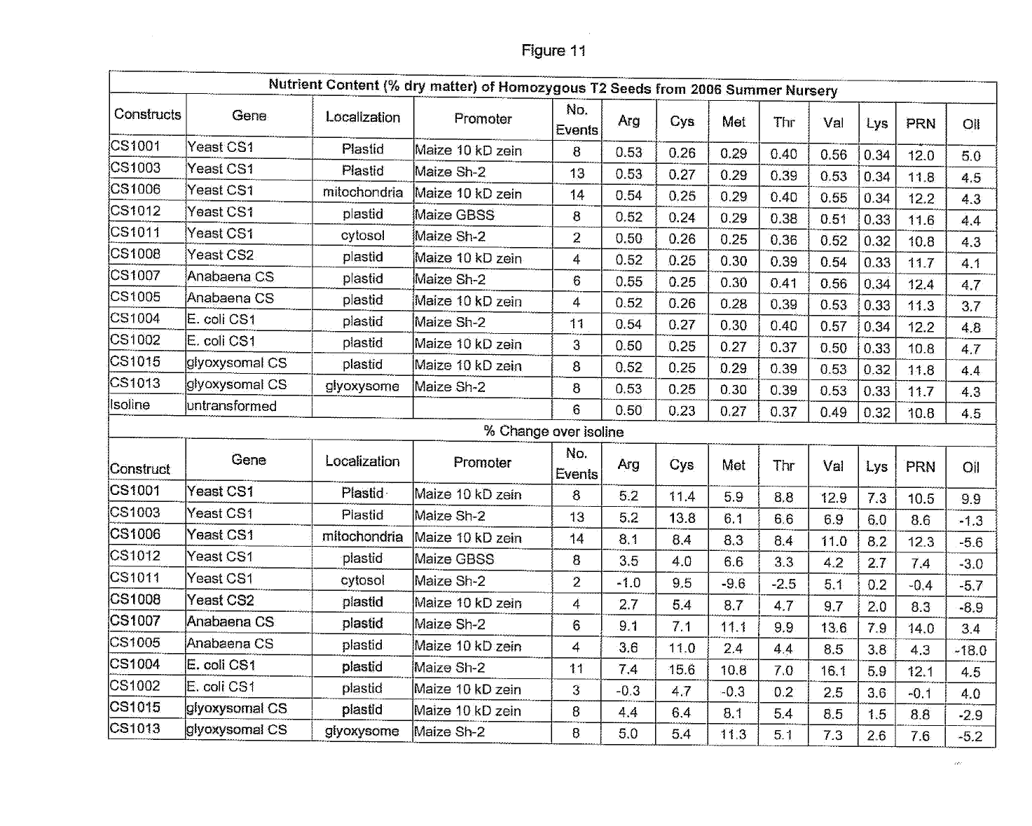

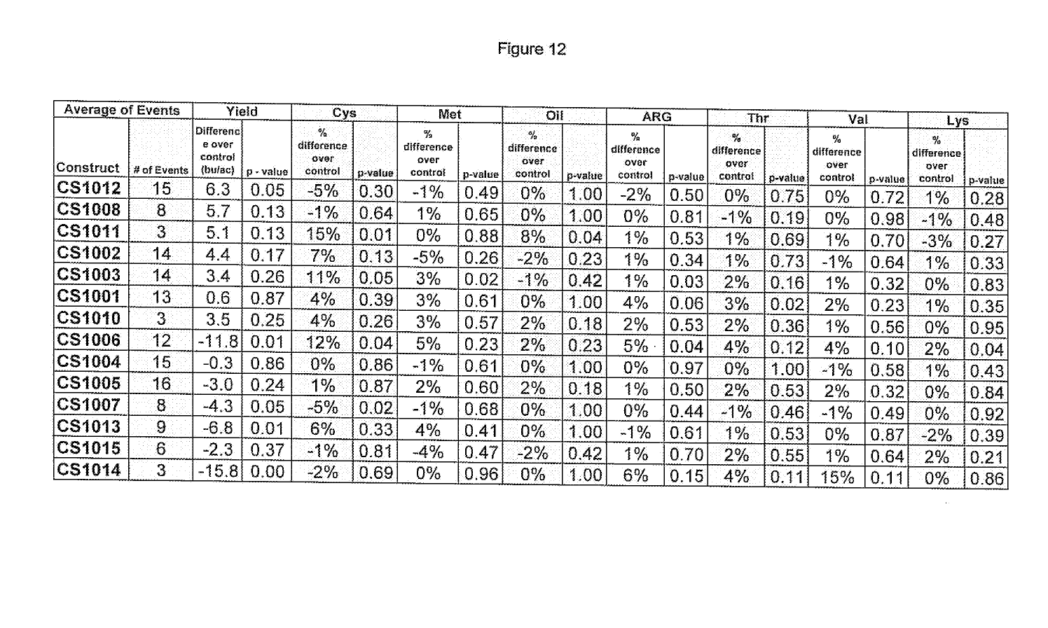

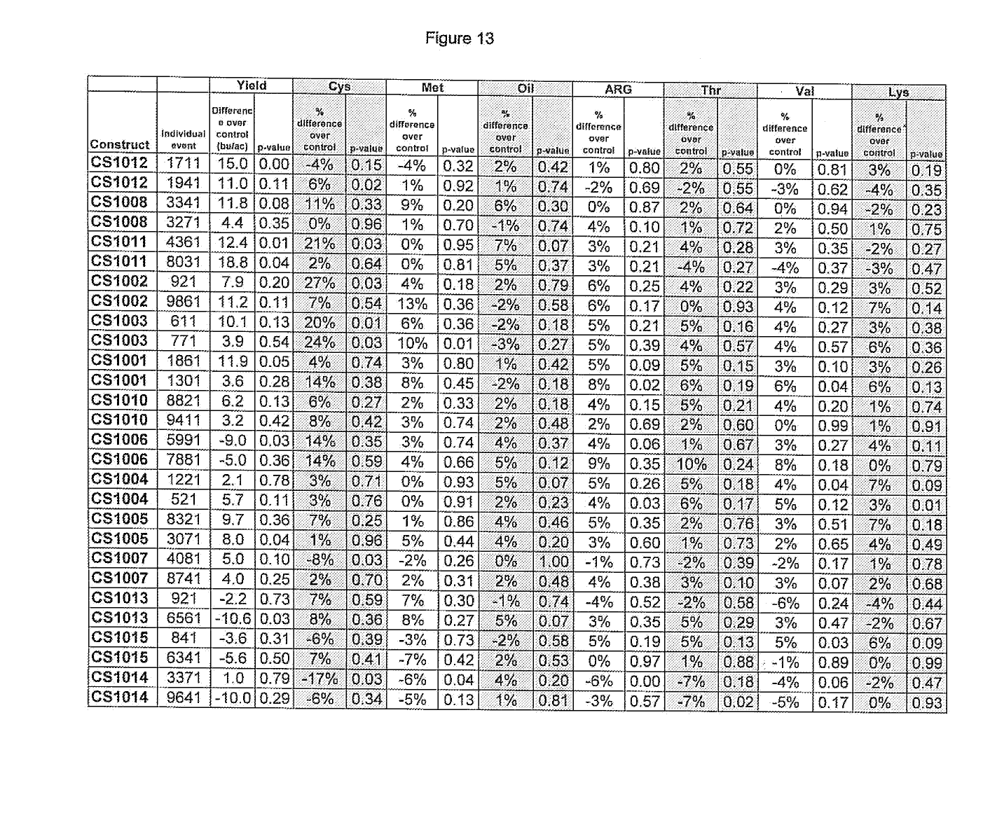

For total amino acid analysis of F2 grain, mature grain samples were ground with an IKA® A11 basic analytical mill (IKA® Works, Inc., Wilmington, N.C.). The samples were re-ground and analyzed for complete amino acid profile (AAP) using the method described in Association of Official Analytical Chemists (AOAC) Official Method 982.30 E (a, b, c), CHP 45.3.05, 2000. Because a commercial event is a single event selected from hundreds of events generated by a large scale transformation of a construct, it is important to look at the performance of that construct not only as an average, but also as individual events. Therefore, data is presented herein as both the average of multiple events from a construct (FIG. 47) as well as two selected single events of the same construct (FIG. 48). As shown in FIGS. 12 and 13, over-expressing yeast CS1 and yeast CS2, E. coli CS1 in an intracellular compartment increased grain yield by at least 3 bushels/acre. The grain composition analysis showed that plants expressing a heterologous CS protein had increased grain yield and/or enhanced grain nutrient contents such as cysteine and methionine.

The results shown in FIGS. 12 and 13 demonstrate the following:

1. Plants expressing an active heterologous CS protein from different organisms such as yeast CS1, yeast CS2 and E. coli CS1 in the plastid of corn seed showed a minimum of about 3 bushels/acre increase in grain yield over wild type control not expressing a heterologous CS.

2. Plants expressing an active CS protein, specifically Yeast CS1 in FIG. 12, in the cytosol (CS1011) of corn seed showed an average of about 5 bushels per acre increase in grain yield and its grain has a about 15% more cysteine and about 8% more oil than isoline control not expressing a heterologous CS.

3. Plants expressing active yeast CS1 protein in the plastid of corn seed (constructs CS1001, CS1003 and CS1012) show in FIG. 13 up to about 15 bushels/acre increase in grain yield or its grain has up to about about 24% increase of cysteine or up to about 10% increase in methionine.

4. Plants expressing active yeast CS1 in the mitochondria (CS1006) showed a significant yield decrease, yet show a significant increase in cysteine. Plants expressing an active glyoxysomeal CS in glyoxysome did not significantly increase grain yield or grain composition.

### Example 7

### Field Test of Transgenic Hybrids

A transgenic corn inbred containing homozygous transgene (CS) was crossed respectively with three proprietary inbred lines (A, B, C) to make F1 hybrid seeds. The transgenic hybrids along with the respective wild type control hybrid were planted in 12 locations with 3 replicates per location for yield test. For grain composition analysis, the transgenic hybrids were planted in 3 locations with 6 replicates per location. Six plants per hybrid were hand-pollinated. Three well-pollinated ears were selected and pooled for grain composition analysis. Oil and protein contents were assayed by NIR methods known to those of skill in the art. See for example, Givens et al (1997) Nutrition Research Reviews 10: 83-114.

For total amino acid analysis of F2 grain, mature grain samples were ground with an IKA® A11 basic analytical mill (IKA® Works, Inc., Wilmington, N.C.). The samples were re-ground and analyzed for complete amino acid profile (AAP) using the method described in Association of Official Analytical Chemists (AOAC) Official Method 982.30 E (a, b, c), CHP 45.3.05, 2000.

Corn is a hybrid crop. The commercial hybrid is developed by crossing one inbred to another inbred from a different heterotic group. There is a strong germplasm interaction that affects heterosis in yield and nutritional quality. Furthermore, there is a strong gene and environmental interaction that affects yield and nutritional quality. Therefore, we evaluated the transgene effect in three hybrids in 12 locations across 4 Midwest State (NE, IA, IL, IN).

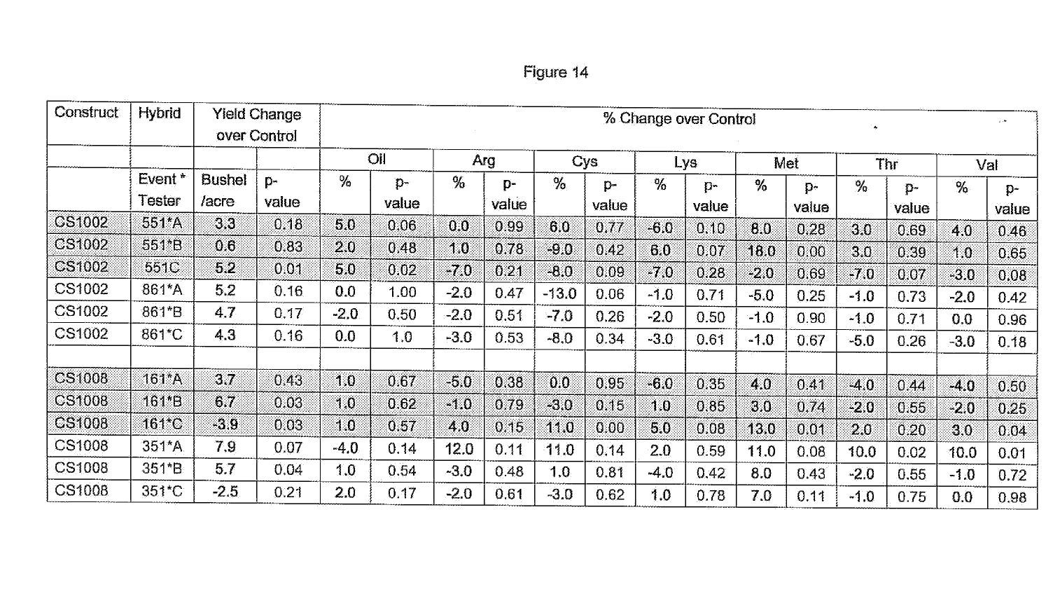

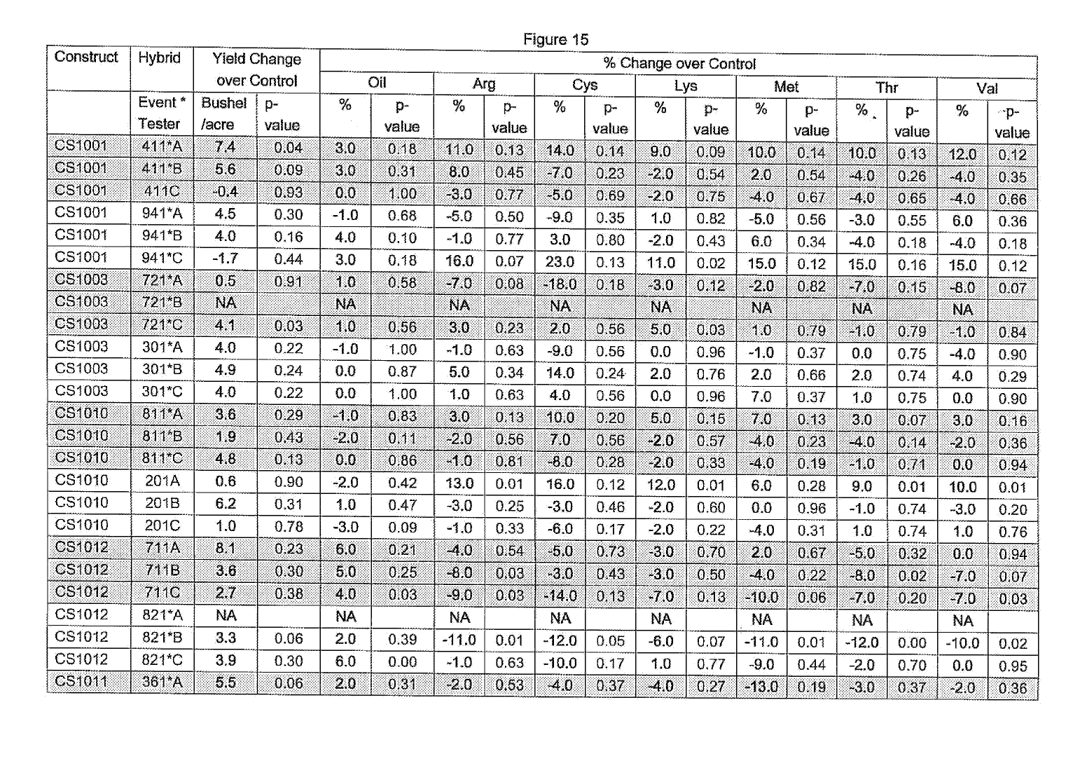

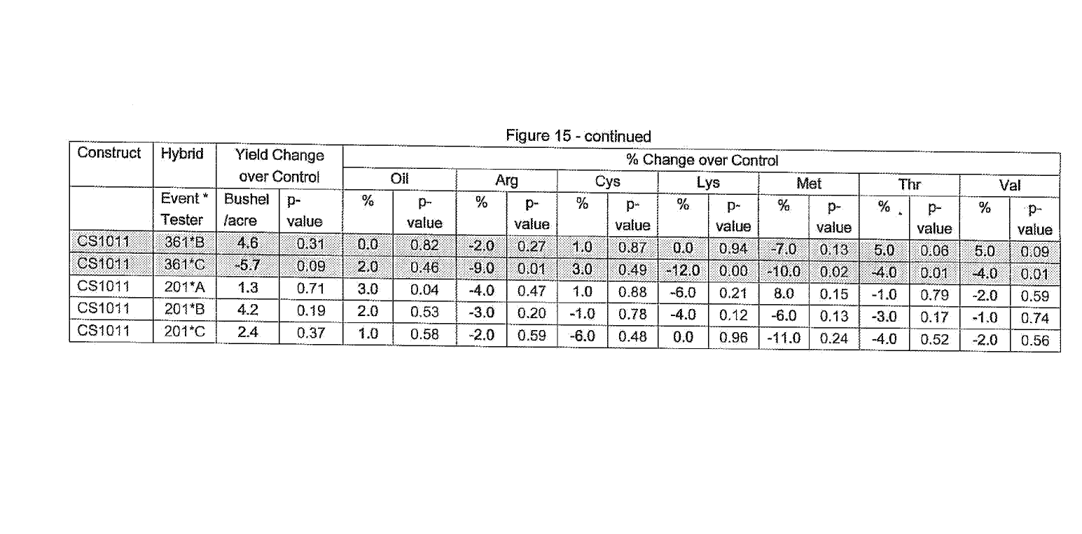

As shown in FIGS. 14 and 15, over-expressing yeast CS1 and yeast CS2, E. coli CS1 in an intracellular compartment increased grain yield by at least 3 bushels/acre. In most cases, the transgenic events expressing a heterologous CS in the seed increase the grain yield by at least 3 bushels per acre in two out of three hybrids tested. In a few cases, the yield was similar between a specific transgenic event and the respective control. This is not unexpected considering the strong interactions between different germplasm and the gene by environmental interactions. Due to heavy rain in Midwest states in June 2008, some of the field plots were flooded and lost. Overall, the data from multiple location and multiple hybrid tests showed that over-expressing yeast CS1 and yeast CS2, E. coli CS1 in an intracellular compartment increased grain yield by at least 3 bushels/acre.

It is known that promoter and gene combinations can affect gene function. Four endosperm preferred promoters were used to drive over-expression of yeast CS1 (FIG. 15). They are maize 10 kD zein promoter, maize Shrunken-2 promoter (ADPGlucose pyrophosphorylase large subunit), maize GBSS promoter (granule bound starch synthase) and maize SSI promoter (starch synthase 1). Although the 10 kD zein promoter and GBSS promoter showed the greater increase in grain yield than Shrunken-2 and SSI promoters when used to drive yeast CS1 over-expression, all four endosperm preferred promoters showed a grain yield increase over control when used to over-express yeast CS1 gene (FIG. 15). The results showed that over-expressing a heterologous CS in seed can increase grain yield by 3 bushels per acre over the control that is not expressing a heterologous CS.

The grain composition analysis showed that plants expressing a heterologous CS protein had similar or greater than control grain nutrient contents such as cysteine and methionine (FIG. 14).

The above examples show that targeting CS expression to the seed, and further in an intracellular compartment, produces valuable traits such as increasing grain yield and/or enhancing the essential amino acids such as cysteine. For example, targeting the expression of heterologous CS, where native CS is not expressed or expressed in a low level, results in grain yield increase and/or enhanced grain composition. Most native CS activity is found in the mitochondria and glyoxysome. The inventors found that targeting the expression of an active heterologous CS in plastid or cytosol of seeds is effective in increasing grain yield and/or increasing grain nutrient content such as the essential amino acid cysteine.

### Example 8

### Stacking CS Events

The above examples show that over-expressing a single CS in an intracellular compartment produces valuable traits such as increasing grain yield or improving nutritional quality. Stacking one CS event with another event or events can lead to further improvement of the traits. The stacking event can be the same heterologous CS expressing at different intracellular compartment or event of a different heterologous CS or events of different genes. For example, the events can be stacked by cross pollination in corn, events expressing yeast CS2 in the plastid can be crossed with events expressing yeast CS1 in the cytosol. Also, for example, events of yeast CS2 can be stacked with E. coli CS1 or events of yeast CS1 can be stacked with events of E. coli CS1 and yeast CS2, respectively. Further, for example, the plant containing both gene events are selfed to produce homozygous seeds containing yeast CS2 and yeast CS1. The stacked events can then be crossed to a tester to make hybrid seeds. The hybrid seeds containing the stacked genes can then be tested in the field to demonstrate the stacking effect on trait performance such as grain yield. In some cases, more than two genes can be stacked to enhance the trait performance. Another way to stack genes is to use a construct stack whereby cloning two or more genes in the same transformation vector or different transformation vectors, the two or more genes are preferably inserted in the same loci, making it easier for trait conversion and commercialization.

The above examples are provided to illustrate the invention but not limit its scope. Other variants of the invention will readily be apparent to one of ordinary skill in the art and are encompassed by the appended claims.

1. A transgenic plant, and its parts, comprising a polynucleotide encoding a heterologous citrate synthase, expressed in the seed or in an intracellular cell compartment of the seed or in the cytosol of the seed, wherein the polynucleotide is selected from the group consisting of:
a) a polynucleotide having a sequence as defined in SEQ ID NO: 1, 2, 3, 4, 5, 6, 7, 8, 9, 10, 11, 12, 13, 14, or 15;
b) a polynucleotide encoding a polypeptide having a sequence as defined in SEQ ID NO: 16, 17, 18, 19, 20, 21, 22, 23, 24, or 25;
c) a polynucleotide having at least 70% sequence identity to a polynucleotide having a sequence as defined in SEQ ID NO: 1, 2, 3, 4, 5, or-6, 7, 8, 9, 10, 11, 12, 13, 14, or 15;
d) a polynucleotide encoding a polypeptide having at least 70% sequence identity to a polypeptide having a sequence as defined in SEQ ID NO: 16, 17, 18, 19, 20, 21, 22, 23, 24, or 25;
e) a polynucleotide hybridizing under stringent conditions to a polynucleotide having a sequence as defined in SEQ ID NO: 1, 2, 3, 4, 5, 6, 7, 8, 9, 10, 11, 12, 13, 14, or 15;
f) a polynucleotide hybridizing under stringent conditions to a polynucleotide encoding a polypeptide having a sequence as defined in SEQ ID NO: 16, 17, 18, 19, 20, 21, 22, 23, 24, or 25; and
g) a polynucleotide complementary to any of the polynucleotides of a) through f).

2. The seed of claim 21, wherein expression of the polynucleotide in the seed confers an economically relevant trait to the seed that is not present or not present at the same level in an isoline.

3. The seed of claim 2, wherein the economically relevant trait is an increase of at least 3 bushels per acre grain yield over the isoline.

4. The plant of claim 1, wherein the polynucleotide has a sequence as defined in SEQ ID NO: 1, 2, 3, 4, 5, or-6, 7, 8, 9, 10, 11, 12, 13, 14, or 15 or the polynucleotide encodes a polypeptide having a sequence as defined in SEQ ID NO: 16, 17, 18, 19, 20, 21, 22, 23, 24, or 25.

5. The plant of claim 1, wherein the polynucleotide has at least 70% sequence identity to a polynucleotide having a sequence as defined in SEQ ID NO: 1, 2, 3, 4, 5, 6, 7, 8, 9, 10, 11, 12, 13, 14, or 15 or the polynucleotide encodes a polypeptide having at least 70% sequence identity to a polypeptide having a sequence as defined in SEQ ID NO: 16, 17, 18, 19, 20, 21, 22, 23, 24, or 25.

6. The plant of claim 1, wherein the plant is a monocot, a dicot, or is selected from the group consisting of maize, wheat, rice, barley, oat, rye, sorghum, banana, ryegrass, pea, alfalfa, soybean, carrot, celery, tomato, potato, cotton, tobacco, pepper, oilseed rape, beet, cabbage, cauliflower, broccoli, lettuce and Arabidopsis thaliana.

7. The plant of claim 1, wherein the plant has an increase of about 3-19 bushels per acre in grain yield over the grain yield of an isoline.

8. An expression vector comprising a seed-preferred transcription regulatory element operably linked to a polynueleotide, wherein the polynucleotide is selected from the group consisting of:
a) a polynucleotide having a sequence as defined in SEQ ID NO: 1, 2, 3, 4, 5, 6, 7, 8, 9, 10, 11, 12, 13, 14, or 15;
b) a polynucleotide encoding a polypeptide having a sequence as defined in SEQ ID NO: 16 17 18, 19, 20, 21, 22, 23, 24, or 25;
c) a polynucleotide having 70% sequence identity to a polynucleotide having a sequence as defined in SEQ ID NO: 1, 2, 3, 4, 5, or-6, 7, 8, 9, 10, 11, 12, 13, 14 or 15;
d) a polynucleotide encoding a polypeptide having at least 70% sequence identity to a polypeptide having a sequence as defined in SEQ ID NO: 16, 17., 18, 19, 20, 21, 22, 23, 24, or 25;
e) a polynucleotide hybridizing under stringent conditions to a polynucleotide having a sequence as defined in SEQ ID NO: 1, 2, 3, 4, 5, 6, 7, 8, 9, 10, 11, 12, 13, 14, or 15;
f) a polynucleotide hybridizing under stringent conditions to a polynucleotide encoding a polypeptide having a sequence as defined in SEQ ID NO: 16, 17, 18, 19, 20, 21, 22, 23, 24, or 25; and
g) a polynucleotide complementary to any of the polynucleotides of a) through f).

9. The expression vector of claim 8, wherein the seed-preferred transcription regulatory element is further operably linked to an intracellular cell compartments targeting sequence.

10. The expression vector of claim 8, wherein the seed-preferred transcription regulatory element is an endosperm-preferred promoter.

11. The expression vector of claim 8, wherein the polynucleotide has a sequence as defined in SEQ ID NO: 1, 2, 3, 4, 5, 6, 7, 8, 9, 10, 11, 12, 13, 14, or 15 or the polynucleotide has at least 70% sequence identity to a polynucleotide having a sequence as defined in SEQ ID NO: 1, 2, 3, 4, 5, 6, 7, 8, 9, 10, 11, 12, 13, 14, or 15.

12. The expression vector of claim 8 wherein the polynucleotide encodes a polypeptide having a sequence as defined in SEQ 1D NO: 16, 17, 18, 19, 20, 21, 22, 23, 24, or 25 or the polynucleotide encodes a polypeptide having at least 70% sequence identity to a polypeptide having a sequence as defined in SEQ ID NO: 16, 17, 18, 19, 20, 21, 22, 23, 24, or 25.

13. A method of producing a transgenic plant comprising an economically relevant trait, wherein the method comprises the steps of:
a) introducing into the plant the expression vector of claim 8, 9, or 10 wherein the polynucleotide encodes a polypeptide that is capable of conferring the economically relevant trait; and
b) selecting a transgenic plant exhibiting the economically relevant trait.

14. The method of claim 13, wherein the economically relevant trait is at least 3 bushels per acre yield increase over bushel per acre yield of an isoline, or is an increase of about 3-19 bushels per acre in grain yield over the grain yield of an isoline.

15. A transgenic plant and its parts produced by the method of claims 13-14.

16. The method of claim 13, wherein the economically relevant trait is selected from the group consisting of:
a) at least 4% increase in oil content over the oil content of an isoline,
b) at least 4% increase in cysteine of the cysteine content of an isoline, and
c) at least 3 bushel per acre yield increase over bushel per acre yield of an isoline.

17. The method of claim 13, wherein the economically relevant trait is an increase of 4%-27% in cysteine content over the cysteine content of an isoline.

18. The method of claim 13, wherein the economically relevant trait is an increase of at least 5% in one or more amino acids selected from the group consisting of threonine, cysteine, valine, methionine, lysine, and arginine, over the amounts of said amino acid in an isoline.

19. The method of claim 13, wherein the economically relevant trait is an increase of 4-10% in oil content in seeds over the oil content in seeds of isoline.

20. A transgenic plant and its parts produced by the method of any of claims 16-19.

21. A seed produced from the plant of claim 1, wherein the seed comprises the polynucleotide of claim 1.

22. The seed claim 2, wherein the economically relevant trait is selected from the group consisting of at least 4% increase in oil content over the oil content of an isoline, at least 4% increase in cycteine of the cysteine content of an isoline, and at least 3 bushel per acre yield increase over bushel per acre yield of an isoline.

23. The seed claim 2, wherein the seed has an increase of at least 5% in one or more amino acids selected from the group consisting of: threonine, cysteine, valine, methionine, lysine, and arginine, over the amounts of said amino acid in an isoline.

24. The seed of claim 2, wherein the seed has an increase of 4%-27% in cysteine content over the cysteine content of an isoline.

25. The plant of claim 2, wherein the plant has an increase of about 3-19 bushels per acre in grain yield over the grain yield of an isoline.

26. The seed of claim 2, wherein the seed has an increase of 2%-13% in methionine content over the methionine content of an isoline.

27. New The seed of claim 2, wherein the seed has an increase of 4%-10% in oil content over the oil content of an isoline.

28. The seed of claim 2, wherein the economically relevant trait is selected from the group consisting of:
a) an increase of at least 3 bushels per acre in grain yield over the isoline;
b) an increase of at least 3 bushels per acre in grain yield over the isoline and the seed has at least 4% more cysteine than the isoline seed;
c) at least 3 bushels/acre increase in grain yield over the isoline and the seed has a at least 4% increase of cysteine and at least 2% increase in methionine than the isoline seed; and
d) at least 3 bushels per acre increase in grain yield over the isoline and the seed has at least 4% more cysteine and at least 4% more oil than the isoline seed,
